(19) United States
(12) Patent Application Publication
(10) Pub. No.: US 2024/0392228 A1
(43) Pub. Date: Nov. 28, 2024

(54) Adjustable Foam Sensor Systems And Related Methods

(71) Applicant: LIFE TECHNOLOGIES CORPORATION, Carlsbad, CA (US)

(72) Inventors: Jason D. Brown, Carlsbad, CA (US); Brycen MILLS, Carlsbad, CA (US)

(21) Appl. No.: 18/696,719

(22) PCT Filed: Sep. 27, 2022
(86) PCT No.: PCT/US2022/044931

§ 371 Date: Mar. 28, 2024

Related U.S. Application Data
(60) Provisional application No. 63/249,950, filed on Sep. 29, 2021.

(51) Int. Cl. C12M 1/21; C12M 1/00; C12M 1/34

(52) U.S. Cl. CPC C12M 41/02; C12M 23/50; C12M 41/44

(57) ABSTRACT

An adjustable foam sensor system is disclosed. The adjustable foam sensor system can include an adjustable foam sensor coupled to a transition member with a foam probe extending from the transition member. The adjustable foam sensor system can also include an adjustable housing that can be coupled to a container interface for interfacing with a container of a fluid processing system. The adjustable housing can be configured to be compressed or extended in response to a force, which in turn can reposition or move the adjustable foam sensor in the container. A controller can automatically adjust the adjustable foam sensor based on sensed and/or determined fluid levels, as well as control the delivery of anti-foam for reducing foam in the container.

### CROSS-REFERENCE TO RELATED APPLICATIONS

The present application claims priority to U.S. Provisional Application No. 63/249,950, filed Sep. 29, 2021, and entitled “Adjustable Foam Sensor Systems and Methods of Suppressing Foam”, the entirety of which is incorporated by reference herein.

### THE FIELD OF THE DISCLOSURE

The present disclosure relates to adjustable foam sensor systems for industrial equipment and processes. More specifically, the present disclosure relates to adjustable foam sensor systems for determining and controlling foam levels generated in bioproduction equipment and biological processes.

### BACKGROUND

The generation of foam in industrial processes is a persistent problem that can affect the purity, clarity, yield and overall quality and effectiveness of the end-product. In the bioproduction industry, a variety of biological processes and equipment generate foam during normal operation. Foam generated in bioproduction equipment can introduce contaminants, clog equipment, degrade cell and growth media, reduce oxygen levels, increase carbon dioxide levels and reduce end-product purity, yield and efficacy. The effects of foam are especially pronounced in the bioproduction and pharmaceutical industry where the degree of purity and yield of the end-product can drastically effect safety, efficacy and profitability.

### SUMMARY OF INVENTION

Current industrial foam sensors suffer from numerous shortcomings and lack versatility necessary for integration across a variety of bioproduction equipment and biological processes. Conventional foam sensors are not readily adjustable, mountable or interchangeable across a diverse set of bioproduction equipment and biological processes. For example, conventional foam sensors cannot detect foam at different vertical and horizontal positions within bioproduction equipment. Conventional foam sensors are also incapable of integration across a diverse set bioproduction equipment and mounting positions. Conventional foam sensors are not modular or equipment agnostic, and therefore, require customization and redesign when mounted at different locations and to different bioproduction equipment.

Modern bioproduction processes utilize a diverse set of bioprocessing equipment, analytical instrumentation and control systems that produce a diverse array of highly purified biological end-products. Current foam sensors are incapable of efficient integration across the full range of current and emerging bioproduction equipment and biological processes.

Therefore, there is a need for improved foam sensor systems and methods of use in industrial equipment and processes, and specifically, in bioproduction equipment and biological processes.

The present disclosure details the components, functionality and operation of adjustable foam sensor systems and methods of suppressing foam in industrial equipment and processes. More specifically, the exemplary adjustable foam sensor systems disclosed herein can detect, control and suppress foam generated in a variety of locations within bioproduction equipment and biological processes

In one aspect, an adjustable foam sensor assembly is described that includes an adjustable housing having a housing base coupled to an adjustable housing body. The adjustable housing body can be compressed or extended in response to a force. The adjustable foam sensor assembly can further include a container interface having an interface opening and an interface surface coupled to the adjustable housing body. Additionally, the adjustable foam sensor assembly can include an adjustable foam sensor having a sensor base coupled to a transition member with a first foam probe tip extending from the transition member. The transition member can extend through the interface opening and can be extended or retracted through the interface opening in response to the force.

In some variations one or more of the following features can optionally be included in any feasible combination. The adjustable foam sensor assembly can further include a sensor probe guide within the adjustable housing body that retains and centers the transition member in the interface opening, and the sensor probe guide can include a guide opening through which the transition member extends. The transition member can be extended or retracted through the guide opening in response to the force. The first foam probe tip can be L-shaped. The adjustable foam sensor assembly can further include a second foam probe tip extending from the transition member. The adjustable foam sensor assembly can further include a third foam probe tip extending from the transition member. The first, second or third foam probe tip can be L-shaped. The first, second or third foam probe tip sensor can be L-shaped.

In another aspect, an adjustable foam sensor assembly is described that includes a container with a first port and an adjustable housing including a housing base coupled to an adjustable housing body. The adjustable housing body can be compressed or extended in response to a force. The adjustable foam sensor assembly can include a container interface having an interface opening and a first interface surface coupled to the adjustable housing body, as well as a second interface surface coupled to the first port. The adjustable foam sensor assembly can further include an adjustable foam sensor including a sensor base coupled to a transition member with a first foam probe tip extending from the transition member. The transition member can extend through the interface opening and can be extended or retracted through the interface opening in response to the force.

In some variations one or more of the following features can optionally be included in any feasible combination. The adjustable foam sensor assembly can further include a sensor probe guide within the adjustable housing body that retains and centers the transition member in the interface opening. The sensor probe guide can include a guide opening through which the transition member extends. The transition member can be extended or retracted through the guide opening in response to the force. The first foam probe tip can be L-shaped. The adjustable foam sensor assembly can further include a second foam probe tip extending from the transition member. The adjustable foam sensor assembly can further include a third foam probe tip extending from the transition member. The first, second or third foam probe tip can be L-shaped.

In another interrelated aspect of the current subject matter, a method of manufacturing an adjustable foam sensor system includes hermetically coupling an adjustable foam sensor assembly to a container with a container port. The foam sensor assembly can include an adjustable housing including a housing base coupled to an adjustable housing body. The foam sensor assembly can further include a container interface including an interface opening, a first interface surface coupled to the adjustable housing body, and a second interface surface coupled to the container port. The foam sensor assembly can also include an adjustable foam sensor including a sensor base coupled to a transition member with a foam probe tip extending from the transition member. The method of manufacturing can further include simultaneously irradiating the adjustable foam sensor and the container.

In yet another interrelated aspect of the current subject matter, a method of suppressing foam in a container can include at least partially filling a container with a fluid. The container can include a first container port coupled to an anti-foam dispenser and a second container port coupled to an adjustable foam sensor assembly. The adjustable foam sensor assembly can include an adjustable housing including a housing base coupled to an adjustable housing body. The adjustable foam sensor assembly can further include a container interface having an interface opening, a first interface surface coupled to the adjustable housing body, and a second interface surface coupled to the container port. The adjustable foam sensor assembly can include an adjustable foam sensor including a sensor base coupled to a transition member with a foam probe tip extending from the transition member. The method of suppressing foam in a container can further include applying a force to the housing base to extend or retract the housing base and extend or retract the transition member through the interface opening and container port.

In some embodiments, the method of suppressing foam in a container can further include detecting the magnitude of foam proximate a top surface of the fluid with the foam probe tip. The method of suppressing foam in a container can further include sending a signal indicative of the magnitude of foam from the foam probe tip to a controller. The method of suppressing foam in a container can further include dispensing an antifoaming agent into the container through the second container port in response to processing the signal at the controller. The dispensing an antifoaming agent can include dispensing an antifoaming agent proximate to the foam probe tip. Detecting and sending the signal can include continuously detecting the magnitude of foam proximate a top surface of the fluid with the foam probe tip and continuously sending the signal indicative of the magnitude of foam from the foam probe tip to a controller over predetermined intervals of time.

In another aspect, an adjustable foam sensor assembly is described that includes a container interface having an interface opening. The container interface can be configured to couple to a container of a fluid processing system. The adjustable foam sensor assembly can include an adjustable foam sensor including a transition member with a first foam probe extending from the transition member. The first foam probe can detect a presence of foam within the container, and the transition member can extend through the interface opening and move along the interface opening in response to a force applied to the adjustable foam sensor to thereby move the first foam probe from a first position to a second position.

In some variations one or more of the following features can optionally be included in any feasible combination. The adjustable foam sensor can further include an adjustable housing configured to transition between a compressed configuration and an extended configuration. The compressed configuration can position the first foam probe in the first position and the extended configuration can position the first foam probe in the second position that is a distance from the first position.

In some embodiments, the adjustable foam sensor assembly can further include a sensor probe guide within the adjustable housing that retains and centers the transition member in the interface opening. The sensor probe guide can include a guide opening through which the transition member extends. The transition member can linearly translate through the guide opening in response to the applied force. The transition member can be coupled to a linear actuator. The transition member can be coupled to a rotary actuator. The transition member can be L-shaped.

In some embodiments, the adjustable foam sensor assembly can further include a second foam probe extending from the transition member. The adjustable foam sensor assembly can further include a third foam probe extending from the transition member. The transition member can be formed of a flexible material. The transition member can be formed of a rigid material. The first foam probe can be configured to communicate with a controller and provide sensed foam data to the controller when the first foam probe is in contact with foam. The sensed foam data can indicate the presence of foam within the container.

In another aspect, an adjustable foam sensor system is described that includes a container of a fluid processing system. The container can be configured to contain a fluid and include a first port. The adjustable foam sensor system including a container interface having an interface opening and a first interface surface coupled to the first port. The adjustable foam sensor system can further include an adjustable foam sensor having a transition member with a first foam probe extending from the transition member. The first foam probe can detect a presence of foam within the container, and the transition member can extend through the interface opening and move along the interface opening in response to a force applied to the adjustable foam sensor to thereby move the first foam probe from a first position to a second position.

In some variations one or more of the following features can optionally be included in any feasible combination. The adjustable foam sensor system can further include an adjustable housing configured to transition between a compressed configuration and an extended configuration. The compressed configuration can position the first foam probe in the first position and the extended configuration can position the first foam probe in the second position that is a distance from the first position. The adjustable foam sensor system can further include a sensor probe guide within the adjustable housing body that retains and centers the transition member in the interface opening. The sensor probe guide can include a guide opening through which the transition member extends.

In some embodiments, the adjustable foam sensor system can further include an anti-foam dispenser coupled to a second port of the container for dispensing anti-foam into the container. The adjustable foam sensor system can further include a fluid measuring device that is at least one of coupled to the container and in communication with the fluid for generating measurement data for determining a fluid level in the container. The fluid measuring device can include a mass measuring sensor or a pressure measuring sensor.

In some embodiments, the adjustable foam sensor system can further include a controller communicatively coupled to the fluid measuring device, the adjustable foam sensor, and the anti-foam dispenser. The controller can be configured to perform operations including receiving measurement data from the fluid measuring device. The measurement data can be associated with the fluid contained in the container. The operations can further include comparing the measurement data against a fluid parameter and activating, in response to the measurement data being different than the fluid parameter, the adjustable foam sensor assembly to move the first foam probe of the adjustable foam sensor assembly from a first position to a second position within the container. The operations can further include receiving sensed foam data from the foam probe indicating the presence of foam within the container and controlling the anti-foam dispenser to deliver a volume of anti-foam solution into the container for reducing the foam.

In some embodiments, the transition member can linearly translate through the guide opening in response to the applied force. The transition member can be coupled to a linear actuator. The transition member can be coupled to a rotary actuator. The transition member can be L-shaped. The adjustable foam sensor system can further include a second foam probe extending from the transition member. The adjustable foam sensor system can further include a third foam probe extending from the transition member. The transition member can be formed of a flexible material. The transition member can be formed of a rigid material. The first foam probe can be configured to communicate with a controller and provide a foam signal for the controller when a probe surface of the first foam probe is in contact with foam. The foam signal can indicate the presence of foam within the container.

In another aspect, a method of manufacturing an adjustable foam sensor system is described that includes hermetically coupling an adjustable foam sensor assembly to a container with a container port. The foam sensor assembly can include a container interface having an interface opening, and the container interface can be configured to couple to a container of a fluid processing system. The adjustable foam sensor system can include an adjustable foam sensor having a transition member with a foam probe extending from the transition member. The foam probe can detect a presence of foam within the container, and the transition member can extend through the interface opening and move along the interface opening in response to a force applied to the adjustable foam sensor to thereby move the foam probe from a first position to a second position. The method of manufacturing can further include simultaneously irradiating the adjustable foam sensor and the container.

In yet another aspect, a method of reducing foam in a container is described that includes at least partially filling a container with a fluid. The container can include a first container port coupled to an adjustable foam sensor assembly, and the adjustable foam sensor assembly can include a container interface having an interface opening. The container interface can be configured to couple to the container. The adjustable foam sensor assembly can include an adjustable foam sensor having a transition member with a foam probe extending from the transition member. The foam probe can sense a presence of foam within the container, and the transition member can extend through the interface opening and move along the interface opening in response to a force applied to the adjustable foam sensor to thereby move the foam probe from a first position to a second position. The method of reducing foam in the container can further include applying a force to the transition member to move the foam probe from the first position to the second position that is above and adjacent a top surface of the fluid.

In some variations one or more of the following can optionally be included in any feasible combination. The method of reducing foam in a container can further include detecting, by the foam probe, the presence of foam within the container. The method of reducing foam in a container can further include receiving, at a controller in communication with the foam probe, a signal indicative of the presence of foam. The method of reducing foam in a container can further include activating, by the controller, an anti-foam dispenser coupled to a second container port to dispense a volume of antifoaming agent into the container to reduce the foam. In some embodiments, the detecting the presence of foam within the container can include continuously sending, from the controller, a signal to the foam probe and identifying a change in the signal indicative of the presence of foam.

In yet another aspect, a method for detecting foam in a fluid processing system is described that can include receiving, at a processor and from a fluid measuring device, measurement data associated with a fluid contained in a container of a fluid processing system. The method for detecting foam in a fluid processing system can further include determining, at the processor and based on the received measurement data, a fluid level. The method for detecting foam in a fluid processing system can further include comparing, at the processor, the determined fluid level against a fluid parameter. The method for detecting foam in a fluid processing system can also include activating, by a controller and in response to the measurement data being different than the fluid parameter, an adjustable foam sensor assembly to move a foam probe of the adjustable foam sensor assembly from a first position to a second position within the container. In some embodiments, the foam probe can be configured to detect a presence of foam within the container.

In some variations one or more of the following can optionally be included in any feasible combination. The method of detecting foam in a fluid processing system can further include receiving, at the processor and from the foam probe, sensed foam data indicating the presence of foam within the container and controlling, by the controller and in response to the sensed foam data, an anti-foam dispenser to deliver a volume of anti-foam solution into the container for reducing the foam. The method of detecting foam in a fluid processing system can further include receiving, at the processor and from the foam probe, sensed foam data indicating the presence of foam within the container and controlling, by the controller and in response to the sensed foam data, a mechanical element of the fluid processing system to cause a reduction in foam.

In some embodiments, the fluid measuring device can include a mass measuring sensor or a pressure measuring sensor. The activating the adjustable foam sensor system can include activating a linear actuator coupled to the foam probe. The activating the adjustable foam sensor system can include activating a rotary actuator coupled to the foam probe. The second position can be above and adjacent a top surface of the fluid. The mechanical element can include a part of a mixer and/or a sparger.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

Various embodiments of the present disclosure are described herein with reference to the appended drawings. It is appreciated that these drawings depict only typical embodiments of the disclosure and are therefore not to be considered limiting of its scope.

FIG. 1 illustrates a side cross-section view of a fluid processing system incorporating an exemplary adjustable foam sensor system.

FIG. 2A illustrates a cross-sectional view of an implementation of the adjustable foam sensor assembly of FIG. 1 shown with a foam probe of the adjustable foam sensor assembly in a first position.

FIG. 2B illustrates a cross-sectional view of an implementation of the adjustable foam sensor assembly of FIG. 1 shown with the foam probe in a second position.

FIG. 3A illustrates a side perspective exploded view of an exemplary adjustable foam sensor assembly with the adjustable sensor housing shown in an extended configuration.

FIG. 3B illustrates a side perspective exploded view of an exemplary adjustable foam sensor assembly with the adjustable sensor housing shown in a collapsed configuration.

FIG. 4A illustrates a side perspective view of an exemplary adjustable foam sensor assembly in an extended configuration.

FIG. 4B illustrates a side perspective view of the adjustable foam sensor assembly of FIG. 4A shown in a collapsed configuration.

FIG. 5A illustrates a schematic side view of an implementation of the adjustable foam sensor assembly including an actuator shown in an extended configuration.

FIG. 5B illustrates a schematic side view of the adjustable foam sensor assembly of FIG. 5A with the actuator shown in a retracted configuration.

FIG. 6 illustrates a side cross-section view of exemplary adjustable foam sensor assemblies integrated with a container of a fluid processing system.

FIG. 7 illustrates a side perspective view of an exemplary adjustable foam sensor assembly including a foam sensor holder.

FIG. 8A illustrates a side view of an exemplary foam sensor holder shown in an extended state.

FIG. 8B illustrates a side view of the exemplary foam sensor holder of FIG. 8A shown in a collapsed state.

FIG. 8C illustrates a top view of the exemplary foam sensor holder of FIG. 8A.

FIG. 9 illustrates communication between hardware of an adjustable foam sensor system according to exemplary embodiments of the present disclosure.

FIG. 10 illustrates an adjustable foam sensor system process according to exemplary embodiments of the present disclosure.

FIG. 11 illustrates a block diagram depicting an example of a computing system consistent with implementations of the current subject matter.

The figures may not be to scale in absolute or comparative terms and are intended to be exemplary. The relative placement of features and elements may have been modified for the purpose of illustrative clarity. Where practical, the same or similar reference numbers denote the same or similar or equivalent structures, features, aspects, or elements, in accordance with one or more embodiments.

### DETAILED DESCRIPTION OF THE PREFERRED EMBODIMENTS

This disclosure is not limited only to the specific and exemplified apparatus, systems, methods, or process parameters disclosed herein. The exemplary apparatus, systems, methods and process parameters may vary as would be understood by one of ordinary skill in the art. Similarly, the terminology used herein is only for the purpose of describing particular embodiments of the present disclosure and is not intended to limit the scope of the disclosure in any manner.

All publications, patents, and patent applications cited herein, whether supra or infra, are hereby incorporated by reference in their entirety to the same extent as if each individual publication, patent, or patent application was specifically and individually indicated to be incorporated by reference.

The term “comprising” which is synonymous with “including,” “containing,” or “characterized by,” is inclusive or open-ended and does not exclude additional, unrecited elements or method steps.

It will be noted that, as used in this specification and the appended claims, the singular forms “a,” “an” and “the” include plural referents unless the content clearly dictates otherwise. Thus, for example, reference to a “partition” includes one, two, or more partitions.

As used in the specification and appended claims, directional terms, such as “top,” “bottom,” “left,” “right,” “up,” “down,” “upper,” “lower,” “proximal,” “distal” and the like are used herein solely to indicate relative directions and are not otherwise intended to limit the scope of the disclosure or claims.

Where possible, like numbering of elements have been used in various figures. Furthermore, multiple instances of an element and or sub-elements of a parent element may each include separate letters appended to the element number. For example, two instances of a particular element “10” or two alternative embodiments of a particular element may be labeled as “10A” and “10B”. In that case, the element label may be used without an appended letter (e.g., “10”) to generally refer to all instances of the element or any one of the elements. Element labels including an appended letter (e.g., “10A”) can be used to refer to a specific instance of the element or to distinguish or draw attention to multiple uses of the element. Furthermore, an element label with an appended letter can be used to designate an alternative design, structure, function, implementation, and/or embodiment of an element or feature without an appended letter. Likewise, an element label with an appended letter can be used to indicate a sub-element of a parent element. For instance, an element “12” can comprise sub-elements “12A” and “12B.”

Various aspects of the present devices and systems may be illustrated by describing components that are coupled, attached, and/or joined together. As used herein, the terms “coupled”, “attached”, and/or “joined” are used to indicate either a direct connection between two components or, where appropriate, an indirect connection to one another through intervening or intermediate components. In contrast, when a component is referred to as being “directly coupled”, “directly attached”, and/or “directly joined” to another component, there are no intervening elements present. Furthermore, as used herein, the terms “connection,” “connected,” and the like do not necessarily imply direct contact between the two or more elements.

Various aspects of the present devices, systems, and methods may be illustrated with reference to one or more exemplary embodiments. As used herein, the term “embodiment” means “serving as an example, instance, or illustration,” and should not necessarily be construed as preferred or advantageous over other embodiments disclosed herein.

Unless defined otherwise, all technical and scientific terms used herein have the same meaning as commonly understood by one of ordinary skill in the art to which the present disclosure pertains. Although methods and materials similar or equivalent to those described herein can be used in the practice of the present disclosure, the preferred materials and methods are described herein.

The present disclosure relates to adjustable foam sensor systems for industrial equipment and processes. More specifically, the present disclosure relates to adjustable foam sensor systems for determining a presence of foam within fluid processing systems, such as along a top surface of a fluid contained in a container of a fluid processing system. The adjustable foam sensor systems can also control foam generated in fluid processing systems and fluid processes, such as reduce and/or eliminate foam within the fluid processing systems. For example, the fluid processing systems and fluid processes can include bioproduction equipment and biological processes, respectively. Biological processes include processes that use or yield biological reagents or end-products including, but not limited to, carbohydrate, cell, cell media, drug, enzyme, lipid, nucleic acid (e.g., DNA, RNA), pharmaceutical, plasmid, protein, reagent, vaccine, viral vector and virus end-products. Bioproduction equipment can include, but is not limited to, bioreactors, cell culture systems and vessels, fermenters, fluid management systems, mixers, storage containers and/or other equipment capable of producing, mixing, managing and storing biological reagents and end-products.

Although the systems disclosed herein are primarily designed for use with biological processes, the apparatus and methods of the present disclosure can also be used with non-biological processes where it is necessary to detect and control the amount of foam generated throughout the process. Such applications can be found in the production of chemicals, gas, medicines, oil and other products. The disclosure and examples set forth herein of adjustable foam sensor systems are generally applicable to industrial equipment and processes.

The exemplary adjustable foam sensor systems provide a hermetically sealed and sterile environment for detecting, sensing, reducing, diffusing and/or eliminating foam in fluid processing systems and fluid processes.

In some embodiments, the adjustable foam sensor systems can include one or more adjustable foam sensor assemblies that can be manually (e.g., by a user) and/or automatically (e.g., by an actuator and controller) adjusted to achieve accurate foam detection. The adjustable foam sensor system can continuously detect and monitor for the presence of foam within the fluid processing system, such as the presence of foam positioned along and/or above a top fluid surface of a fluid contained in a container of the fluid processing system. In some embodiments, the one or more adjustable foam sensors can collect sensed foam data, such as whether or not foam is in contact with an adjustable foam sensor. The adjustable foam sensors can be positioned in a variety of locations within a container containing fluid, as well as positioned across a variety of fluid processing equipment (e.g., bioprocessing equipment).

In some embodiments, the adjustable foam sensor system can be in communication with and/or include a controller that receives the sensed foam data. The controller can include at least one processor and memory and can analyze the received sensed foam data from one or more adjustable foam sensors of the adjustable foam sensor system. For example, the controller can analyze the received sensed foam data and identify when a foam probe of an adjustable foam sensor detects foam (e.g., the foam probe comes into contact with foam). For example, the foam probe can include a resistive and/or capacitive type sensor that provides a change in sensed foam data (e.g., change in electrical resistance and/or capacitance) as a result of the foam probe coming into contact with foam, thus indicating a presence of foam within the container (e.g., above and/or along the top fluid surface). Other sensing mechanisms can be implemented in the foam probe for collecting the sensed foam data the controller can use to determine whether foam is present in the container, including completing a circuit (e.g., through the foam and/or fluid), etc.

In some embodiments, the foam probe can sense and/or detect the presence of foam along and/or above the top surface of fluid and/or within the container containing the fluid and send the sensed foam data indicating the presence of foam to the controller. Once the controller determines the foam probe has detected the presence of foam, the controller can perform one or more actions to reduce and/or eliminate the foam. For example, the controller can activate an anti-foam dispenser to dispense a volume of anti-foaming agent in the container. The anti-foaming agent can be dispensed into the fluid processing equipment (e.g., container) to reduce and/or eliminate foam. In some embodiments, the adjustable foam sensor system can include various elements, such as more than one adjustable foam sensor positioned at various positions within the container for determining various aspects of the detected foam, such as an approximate volume of the foam, an approximate thickness of the foam, and/or an approximate coverage of the foam along the top surface of the fluid and/or within the container.

In some embodiments, the controller can be in communication with one or more mechanical elements of the adjustable foam sensor system and/or fluid processing system, and any one of the mechanical elements can be activated to assist with reducing, dispersing, and/or eliminating foam in the fluid processing system. In some embodiments the controller can be in communication with one or more anti-foam dispensers and one or more mechanical elements to allow the controller to activate either the anti-foam dispenser or mechanical element, as needed, to reduce and/or eliminate foam in the fluid processing system (e.g., container).

For example, the mechanical element can include a foam breaker device (e.g., for breaking down foam into fluid and thus reducing foam), a fluid mixing device (e.g., for mixing fluid in fluid processing system), or a sparger (e.g., for delivering gas into the fluid of fluid processing system). As such, the controller, for example, can reduce the mixing speed of the fluid mixing device and/or reduce sparging in order to reduce foam formation.

The exemplary adjustable foam sensor systems can automatically and continuously monitor one or more locations within the fluid processing system for detecting the presence of foam and, as a result of detecting the presence of foam, can reduce and/or eliminate foam within the fluid processing system. By efficiently and effectively reducing and/or eliminating foam in the fluid processing system, the adjustable foam sensor systems described herein can prevent adverse process conditions and clogging of equipment. The reduction and/or elimination of foam can prevent equipment clogging, contamination and degradation of cell and growth media, increase dissolved oxygen levels, decrease carbon dioxide levels and increase end-product purity, yield and efficacy.

Exemplary adjustable foam sensor systems disclosed herein can be incorporated with and/or mounted to a variety of different fluid processing systems (e.g., bioproduction equipment) and mounting locations to both detect foam and regulate foam volume and formation at one or more of a variety of locations within the fluid processing system and fluid process. For example, one or more adjustable foam sensor assemblies of an embodiment of an adjustable foam sensor system can be mounted to a variety of locations of a container of the fluid processing system, such as a top container wall, a sidewall, and/or a bottom container wall of the container containing the fluid.

Exemplary adjustable foam sensor systems disclosed herein include an adjustable foam sensor and an adjustable housing, both of which can be extended and retracted to position the sensing probe of the adjustable foam sensor system for detecting at least the presence of foam in more than one location within the fluid processing system. For example, in typical fluid processes, fluid levels within the fluid processing system (e.g., container) may vary over the course of the process. As the level of fluid within the fluid processing system rises or falls and foam is generated on or close to the top surface of the process fluid, the adjustable foam sensors described herein can be repositioned (e.g., retracted or extended) to detect foam at varying fluid levels. This can improve the accuracy and effectiveness of the adjustable foam sensor systems to detect and reduce foam compared to at least some currently available foam sensors.

In some embodiments, the adjustable housing includes a foam sensor positioning mechanism that can me manually, electronically, and/or mechanically controlled. The foam sensor positioning mechanism can be a part of the adjustable housing, contained in the adjustable housing, coupled to the adjustable housing, and/or separate from the adjustable housing. For example, some embodiments of the foam sensor positioning mechanism of the adjustable housing include a telescoping mechanism that can be controlled by a user to cause the foam probe of the adjustable foam sensor to move from at least a first position to a second position within the chamber of the fluid processing system where the second position is a preferred location for detecting foam (e.g., above and adjacent the top fluid surface).

In some embodiments, the foam sensor positioning mechanism of the adjustable housing includes an actuator that is controlled by the controller of the adjustable foam sensor system. For example, the actuator can include a linear or rotary actuator that is coupled to the adjustable foam sensor to cause the foam probe of the adjustable foam sensor to move from at least a first position to a second position within the chamber of the fluid processing system where the second position is a preferred location for detecting foam (e.g., above and adjacent the top fluid surface).

In some embodiments, the foam sensor positioning mechanism automatically adjusts the position of the foam probe of the adjustable foam sensor, such as in response to a change in fluid level (e.g., an increase or decrease in fluid level in the container). Such change in fluid level can place the position of the foam probe in a disadvantaged location for effectively detecting foam along the top surface of the fluid if the foam probe is not repositioned. For example, an increase in fluid level can submerge the foam probe or a decrease in fluid level can increase a distance between the foam probe and the top surface of the fluid such that an unwanted excess of foam would have to form along the top fluid surface before the foam probe would be able to detect the foam. As such, embodiments of the adjustable foam sensor system can include a controller that automatically adjusts the position of the foam probe in response to a determined change in fluid level. This can increase the efficiency and effectiveness of the adjustable foam sensor system to detect foam, such as during fluid processing where fluid levels can change throughout the process.

In some embodiments, the adjustable foam sensor assembly can include at least one fluid measuring device that is in communication with the controller and assists with detecting an increase and/or decrease in fluid level (e.g., due to a change in fluid volume). For example, a fluid measuring device can include a mass measuring sensor or a pressure measuring sensor positioned along and/or within the container containing the fluid. Sensed measurement data from the fluid measuring device (e.g., mass measurement data, pressure measurement data) can be received by the controller and used to determine any changes in fluid volume and/or fluid levels. For example, when the controller determines changes in the fluid volume and/or fluid levels, the controller can automatically and/or approximately automatically activate the foam sensor positioning mechanism (e.g., linear actuator, rotational actuator, etc.) to cause the foam probe of the adjustable foam sensor to be repositioned within the container, such as reposition the foam probe so that it is above and adjacent the top fluid surface in order to effectively sense and detect foam above and/or along the top fluid surface within the chamber.

Exemplary adjustable foam sensor systems disclosed herein are capable of detecting signal fouling and can thereby reduce and/or avoid excessive deployment and use of anti-foaming agents.

Exemplary adjustable foam sensor systems disclosed herein can include a variety of foam probe configurations, including foam probes with different shapes and multiple foam probes per adjustable foam sensor, which can enhance the ability of the adjustable foam sensor systems to detect foam at different and hard-to-reach locations within fluid processing systems. For example, in some biological processes and bioproduction equipment, including some bioreactor, fermenters and mixers, foam can build-up in the corners, near walls or in crevasses of the equipment (e.g., fluid containers). Exemplary adjustable foam sensors can be adjusted to reach various areas (e.g., container corners, adjacent and/or along container walls, and/or in crevasses of the equipment) to detect, measure, reduce and/or eliminate foam.

Exemplary adjustable foam sensor systems herein can also be rotated (e.g., adjustable foam sensor can be rotated by a rotary actuator) within the fluid processing system to position one or more foam probes and/or transition members of the adjustable foam sensor in hard-to-reach locations, corners, under ledges and other locations within the fluid processing system. As such, in addition to up, down and sideways actuation, exemplary adjustable foam sensors can be rotated within the fluid processing system.

In exemplary embodiments, the foam probe and/or transition member of the adjustable foam sensor can be L-shaped and translationally adjusted (e.g., towards and away from a container wall) within a fluid container and/or rotated within the container to detect foam at different locations within the container. In other exemplary embodiments, the adjustable foam sensor assembly includes multiple foam probes and/or transition members of different shapes, including one or more straight and L-shaped foam probe and/or transition member. The multiple foam probes can be translationally moved, such as towards and away from a chamber wall, and/or rotated within the chamber to thereby detect foam at different locations within the fluid processing system.

Exemplary adjustable foam sensor systems disclosed herein can also include a sensor probe guide positioned proximate to and/or coupled, attached or connected to an interface of a container of the fluid processing system. The sensor probe guide can center and retain the adjustable foam sensor, including a transition member and foam probe, such as in a fixed radial position within an adjustable sensor housing and within a chamber port opening of a container. The sensor probe guide can reduce and/or eliminate occurrences of the adjustable foam sensor, including a transition member and foam probe, touching unwanted surfaces. The sensor probe guide can also reduce or eliminate occurrences of the adjustable foam sensor, including a transition member and foam probe, from contacting unwanted moisture droplets or the side wall of container, which can lead to false signals and false foam measurement readings. Exemplary configurations with sensor probe guides can also prevent bridging, which occurs when fluid within the fluid process bridges gaps between the adjustable foam sensor, foam probe and/or transition member and another surface causing potential fouling.

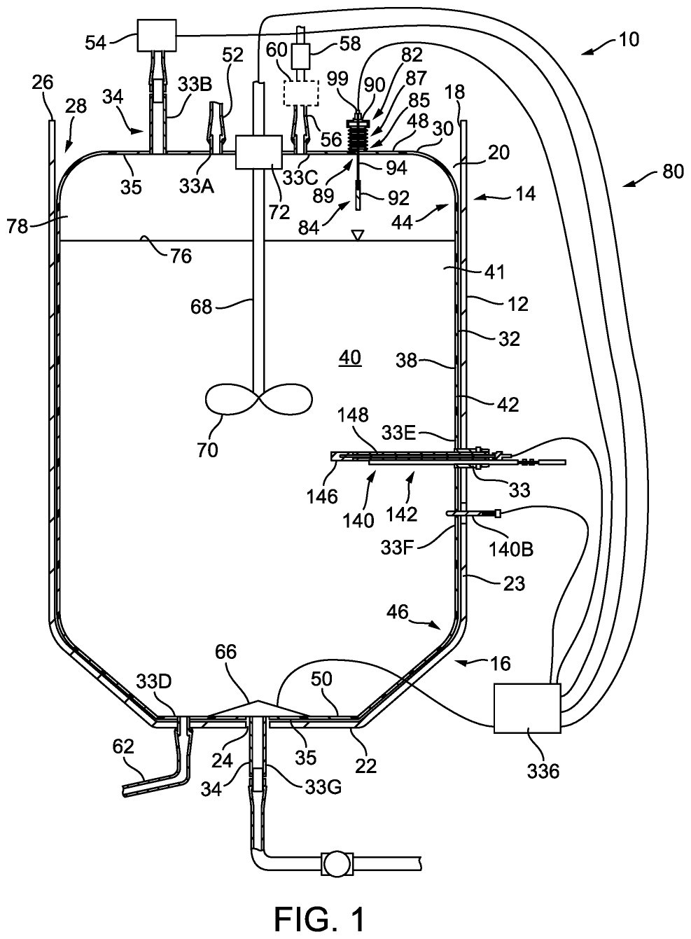

FIG. 1 depicts a fluid processing system 10 (e.g., bioproduction equipment) incorporating an exemplary adjustable foam sensor system 80. In this example, fluid processing system 10 can function as a bioreactor, fermenter, mixer, storage container or fluid management system capable of producing, mixing, managing and storing biological reagents and/or end-products. Fluid processing system 10 can also be used in the production of chemicals, beverages, food products, or others processes where foam regulation is necessary. Fluid processing system 10 can be formed from a substantially rigid support housing 12 in which a container system 30 is disposed. Support housing 12 can include an upper end 14, a lower end 16, and an interior surface 18 that bounds a compartment 20. A floor 22 is formed at lower end 16 of the support housing 12. An encircling sidewall 23 extends up from floor 22 toward upper end 14. One or more openings 24 can extend through the floor 22 and/or sidewall 23 of support housing 12 so as to communicate with compartment 20. Upper end 14 can terminate at a lip 26 that bounds an access opening 28 to compartment 20. If desired, a cover can be hingedly or removably mounted on upper end 14 of the support housing 12 so as to cover all or part of access opening 28.

Support housing 12 can come in a variety of different sizes, shapes, and configurations. An access port can be formed on support housing 12, such as on sidewall 23 or floor 22, to permit manual access to compartment 20. The access port can be selectively closed by a door. Support housing 12 is typically made of metal, such as stainless steel, but other rigid or semi-rigid materials can also be used.

The container system 30 is at least partially disposed within compartment 20 of support housing 12 and is supported thereby. Container system 30 includes a container 32 having a plurality of tube ports 33 mounted thereon. In exemplary embodiments, container 32 is or includes a flexible bag or fluid container (e.g., bioprocess container) having an interior surface 38 that bounds a chamber 40 suitable for holding a fluid 41. For example, the container 32 can include one or more container walls, such as a sidewall 42 that, when the container 32 is inflated and/or at least partly filled with a fluid, has a substantially circular or polygonal transverse cross section that extends between a first end 44 and an opposing second end 46. First end 44 can terminate at a top end wall 48 while second end 46 terminates at a bottom end wall 50. Fluid 41 can include one or more reagents, biological end-products, a biological component used to make a reagent or biological end-product, a culture, or other fluid used in a bioproduction and/or fluid process.

Container 32 is manufactured from and/or includes a flexible, water impermeable material such as a low-density polyethylene or other polymeric sheets or films having a thickness in a range between about 0.1 mm to about 5 mm with about 0.2 mm to about 2 mm being more common. Other thicknesses can also be used. The material can be single ply material or can comprise two or more layers, which are either sealed together or separated to form a double wall container. Where the layers are sealed together, the material can comprise a laminated or extruded material. The laminated material comprises two or more separately formed layers that can be secured together, such as by an adhesive.

The extruded material of the container 32 can include a single integral sheet that comprises two or more layers of different material that are each separated by a contact layer. In some embodiments, the layers can be simultaneously co-extruded. One example of an extruded material that can be used to manufacture the container 32 is the Thermo Scientific CX3-9 film available from Thermo Fisher Scientific. The Thermo Scientific CX3-9 film is a three-layer, 9 mil cast film produced in a cGMP facility. The outer layer is a polyester elastomer coextruded with an ultra-low-density polyethylene product contact layer. The container 32 can also be manufactured from extruded Thermo Scientific CX5-14 cast film also available from Thermo Fisher Scientific.

The material of construction of the container 32 can maintain a sterile environment and surface that can come in direct contact with biological reagents or end-products without degradation of the container 32, biological reagents or end-products. The containers 32 and materials of construction can be sterilized with ionizing radiation or otherwise before, during or after construction. Examples of materials that can be used to manufacture containers 32 used across different biological processes are disclosed in U.S. Pat. No. 6,083,587 which issued on Jul. 4, 2000 and US Patent Publication No. US 2003/0077466 A1, published Apr. 24, 2003 which are each hereby incorporated by specific reference.

In exemplary embodiments, the container 32 is made from a two-dimensional pillow style bag, wherein two sheets of material are placed in overlapping relation and the two sheets are bounded together at their peripheries to form internal chamber 40. Alternatively, a single sheet of material can be folded over and seamed around the periphery to form internal chamber 40. In another embodiment, the container 32 can be formed from a continuous tubular extrusion of polymeric material that is cut to length and the ends seamed closed.

In still other embodiments, container 32 can comprise a three-dimensional bag with an annular sidewall, a two-dimensional top end wall 48 and a two-dimensional bottom end wall 50. This three-dimensional container 32 comprises a plurality of discrete panels, typically three or more, and more commonly four or six. Each panel is substantially identical and makes-up a portion of the sidewall 42, top end wall 48, and bottom end wall 50 of the container 32. Corresponding perimeter edges of each panel can be seamed together. The seams are typically formed using methods known in the art such as heat energies, RF energies, sonics, or other sealing energies.

In alternative embodiments, the panels can be formed in a variety of different patterns. Additional methods of manufacturing three-dimensional bags are disclosed in US Patent Publication No. US 2002/0131654 A1, published Sep. 19, 2002 which is hereby incorporated by specific reference.

Container 32 is typically sterilized so that interior surface 38 and chamber 40 are sterile prior to delivering fluid 41 into chamber 40. It is appreciated that container 32 can be manufactured to have virtually any desired size, shape, and configuration. For example, container 32 can be formed having chamber 40 with a volume that is greater than, less than, or substantially equal to 10 liters, 30 liters, 100 liters, 250 liters, 500 liters, 750 liters, 1,000 liters, 1,500 liters, 3,000 liters, 5,000 liters, 10,000 liters or other desired volumes. The size of the chamber 40 can also be in the range between any two of the above volumes. Although container 32 can be any shape, in exemplary embodiments container 32 is specifically configured to be complementary or substantially complementary to compartment 20 of support housing 12. It is desirable that when container 32 is received within compartment 20, container 32 is generally uniformly supported by support housing 12. Having at least generally uniform support of container 32 by support housing 12 can help to preclude failure of container 32 by hydraulic forces applied to container 32 when filled with fluid. Accordingly, bioprocess containers 32 can be formed to have the same or similar shape as the support housing 12.

Although in the above discussed embodiment container 32 is depicted and discussed as a flexible bag, in alternative embodiments it is appreciated that container 32 can comprise any form of collapsible container or semi-rigid container. Container 32 can also be transparent or opaque and can have ultraviolet light inhibitors incorporated therein.

As depicted in FIG. 1, a plurality of tube ports 33 are in fluid communication with the chamber 40 and are mounted on sidewall 42, top end wall 48, and bottom end wall 50 of the bioprocess container 32. Each tube port 33 typically includes a tubular stem 34 that passes through a hole along container 32 and an annular flange 35 that encircles and radially outwardly projects from stem 34. Flange 35 is welded to interior surface 38 of container 32 to seal closed the opening through which stem 34 passes. It is appreciated that any number of tube ports 33 can be present depending on the intended use of container 32. Tube ports 33 can also be a variety of different types, sizes and configurations. For example, tube ports 33 can be rigid or flexible and stem 34 can be formed having a substantially cylindrical configuration or formed with an outwardly encircling barb. One example of a tube port that can be used is disclosed in U.S. Pat. No. 7,879,599, issued Feb. 1, 2011 and incorporated herein in its entirety by specific reference.

Each tube port 33 can serve a different purpose depending on the type of processing to be undertaken. For example, tube port 33A is mounted on top end wall 48 and is coupled with a fluid line 52 for dispensing media, cultures, nutrients, reagents, biological end-products, components and/or other types of fluids and additives into chamber 40 of container 32. Tube port 33B can also be mounted on top end wall 48 and coupled to an anti-foam dispenser 54 that can be activated to dispense a predetermined and/or calculated (e.g., by a processor) volume and/or flow rate of anti-foaming agent into chamber 40 of container 32.

Tube port 33C is mounted on top end wall 48 and is coupled to one or more exhaust gas filters 58, either directly or through a gas exhaust line 56. Filter 58 enables gas to exit out of container 32 while preventing any contaminates from entering container 32. Filter 58 can also be used to remove any contaminates and/or moisture from the exhaust gas as it passes through filter 58. One example of a filter that can be used is a sterilizing filter that can remove contaminates down to 0.2 microns. Other filters can also be used.

More specifically, filter 58 comprises a porous material through which gas can pass but through which unwanted contaminants, such as bacteria and microorganisms, cannot. The porous material is typically hydrophobic which helps it to repel liquids. For example, filter 58 can be comprised of polyvinylidene fluoride (PVDF). Other materials can also be used. For example, where the system is acting as a bioreactor or fermentor, filter body 58, or the porous material thereof, typically needs to operate as a sterilizing filter and will thus typically have a pore size of 0.22 micrometers (μm) or smaller. The term “pore size” is defined as the largest pore in the material through which a particle can pass. Commonly, the porous material of filter 58 has a pore size in a range between 0.22 and 0.18 μm. However, for pre-filtering applications or for non-sterile applications, the porous material for filter 58 can have a larger pore size, such as in a range between about 0.3 and 1.0 μm. In still other applications, the pore size can be greater than 1.0 μm. One example of filter 58 is the DURAPORE 0.22 μm hydrophobic cartridge filter produced by Millipore. Another example is the PUREFLO UE cartridge filter available from ZenPure.

If desired, a condenser 60 can be disposed between port 33C and filter 58 so that the exhaust gas passes through condenser 60. Condenser 60 can be used to remove moisture from the exhaust gas before the exhaust gas reaches filter 58. Condenser 60 thus helps to remove moisture that can clog filter 58. The condensed moisture can either be returned to container 32 or separately collected. Exemplary condensers 60 and associated components are disclosed in U.S. Pat. No. 8,455,242, issued Jun. 4, 2013, which is incorporated herein in its entirety by specific reference. Other exemplary filters 58 and condensers 60 are disclosed in U.S. patent application Ser. No. 14/588,063, filed Dec. 31, 2014, which is incorporated herein in its entirety by specific reference. Other condensers and filters can also be used.

Tube port 33D is mounted on bottom end wall 50 and is coupled to a drain line 62. Drain line 62 can be used for sampling or otherwise dispensing fluid 41 from container 32. Tube ports 33E and 33F are also depicted as coupled with container 32 on sidewall 42. In addition to those depicted, other tube ports can also be mounted on container 32 for achieving other desired functions. For example, when container 32 is used as a reactor for growing cells or microorganisms, other tube ports 33 can be used to attach various probes such as temperature probes, pressure probes, flow meters, pH probes, dissolved oxygen probes, and the like to container 32.

As depicted in FIG. 1, a sparger 66 is mounted on container 32 for delivering controlled types and quantities of gases to fluid 41 within container 32. This gas passes out through gas filter 58. Sparger 66 can come in a variety of different sizes, shapes, and configurations and can be either secured to or freely resting on or disposed within container 32. One or more spargers 66 can be used and, depending on their function, may emit fine bubbles of gas, larger bubbles of gas, or combinations thereof. The gas that is emitted is typically air, oxygen, nitrogen, or combinations thereof but other gases can also be used. Exemplary spargers 66 are disclosed in U.S. Pat. No. 7,384,783, issued Jun. 10, 2008, US Patent Publication No. 2006/0270036, published Nov. 30, 2006, and US Patent Publication No. 2013/0082410, published Apr. 4, 2013 which are incorporated herein in their entirety by specific reference. Other spargers can also be used.

In exemplary embodiments, sparger 66 is formed from a stem 34 interfacing and/or connected to a gas permeable material fixed to a flange 35 of a tube port 33G. Gas delivered though the stem 34 is forced to travel out through the gas permeable material that can sparge or distribute gas bubbles through fluid 41. Additional examples of gas permeable materials and spargers capable of sparging gas are also disclosed in the above referenced US Patent Publication No. 2006/0270036.

Although not required, certain fluid processing systems 10 and/or containers 32 can include a mixer disposed within the fluid-filled chamber 40. By way of example and not by limitation, the mixer can include a drive shaft 68 with impeller 70, or other mixing element, that projects into chamber 40 through a dynamic seal 72. External rotation of drive shaft 68 facilitates rotation of impeller 70 or other mixing element, which mixes and/or suspends fluid 41 within chamber 40. Sparger 66 is typically disposed directly below or proximate to the mixer in a position where mixing promotes entrainment of the gas bubbles within fluid 41. As shown in FIG. 1, the controller 336 can be in communication with the sparger 66 and/or the drive shaft 68, such as in order to control the sparger 66 (e.g., reduce and/or increase sparging) and/or the drive shaft 68 (e.g., decrease and/or increase rotational speed of drive shaft 68).

In exemplary embodiments, the mixer can include a flexible tube disposed within chamber 40 having a first end coupled to container 32 by a sealed bearing and an opposing second end having an impeller or other mixing element mounted thereon. A drive shaft can be selectively passed down the tube and coupled to the impeller so that rotation of the drive shaft rotates the impeller for mixing fluid 41, but the drive shaft does not directly contact fluid 41. In other embodiments, drive shaft 65 can be configured to repeatedly raise and lower a mixing element attached thereto for mixing fluid 41. Alternatively, or in combination with other mixers, a magnetic stir bar can be disposed within compartment 40 of container 32 and rotated by a magnetic driver disposed outside of container 32. Alternatively, or in combination with other mixers, a stir bar, paddle, or similar mixer can be disposed and project into compartment 40 of container 32. The stir bar can be pivoted, swirled or otherwise moved to mix fluid 41. In addition, mixing can be accomplished by circulating fluid through chamber 40, such as by using a peristaltic pump to move fluid 41 into and out of chamber 40 through a tube having opposing ends sealed to container 32. Any one or more of the mixer embodiments can be controlled by the controller 336. Gas bubbles can also be passed through the fluid to achieve the desired mixing. Finally, support housing 12 and container 32 can be pivoted, rotated or otherwise moved to mix the fluid within container 32. Other conventional mixers and mixing techniques can also be used in fluid processing system 10.

Specific examples of mixers incorporated with flexible bag and/or bioprocess containers 32 are disclosed in U.S. Pat. No. 7,384,783, issued Jun. 10, 2008; U.S. Pat. No. 7,682,067, issued Mar. 23, 2010; and US Patent Publication No. 2006/0196501, issued Sep. 7, 2006 which are incorporated herein by specific reference.

Exemplary adjustable foam sensor systems 80 herein disclosed can be incorporated with and mounted to a variety of different fluid processing systems 10 and mounting locations to both detect and regulate foam buildup within the fluid processing system 10, process chamber 40, container 32 and/or other locations within the fluid processing system 10 or flow lines. Exemplary fluid processing systems 10 can function as a bioreactor, fermenter, mixer, storage container or fluid management system capable of producing, mixing, managing and storing fluid 41 comprising biological reagents, end-products, cultures of living cells, microorganisms and/or other biological or non-biological components. The fluid 41 can include a top surface 76 disposed within chamber 40 with a gap or head space 78 formed between the top surface 76 and top end wall 48 of the container 32.

To oxygenate cells and/or microorganisms within fluid 41 and to otherwise regulate the chemistry within fluid 41, gas is sparged into fluid 41 through sparger 66 while the fluid within container 32 is being mixed, such as using impeller 70. A surfactant can be added to the culture to limit unwanted shear forces on the cells or microorganisms caused by the impeller 70 or other mixing element. The sparged gas bubbles can pass up through fluid 41 and then enter gap 78 as a humid exhaust gas. The exhaust gas can pass out of gap 78 through tube port 33C and eventually exit into the environment through exhaust gas filter 58. As previously discussed, the exhaust gas can also pass through condenser 60 if needed before passing through filter 58. Due to one or more factors, process parameters and/or process environmental conditions, foam can form at, near or above the top surface 76 of the fluid 41. For example, a combination of the surfactant, the waste from the cells/microorganisms, and/or the sparging bubbles passing through the fluid 41 or culture can cause foam to progressively build up on the top surface 76 of fluid 41. If the foam is not reduced and/or eliminated, the foam can eventually pass out through tube port 33C with the exhaust gas and enter and clog the filter 58. The foam can also degrade the process and culture and/or reduce or increase pH, oxygen or carbon dioxide levels beyond optimal process limits. Once the filter 58 becomes clogged by the foam, fluid processing system 10 can be rendered inoperable, requiring process and system shut down. As a result, the culture within the container 32 can die. The foam can also produce buildup and blockage within the condenser 60 and other process components downstream of tube port 33C. Foam in general can have many deleterious effects on a biological and/or industrial process.

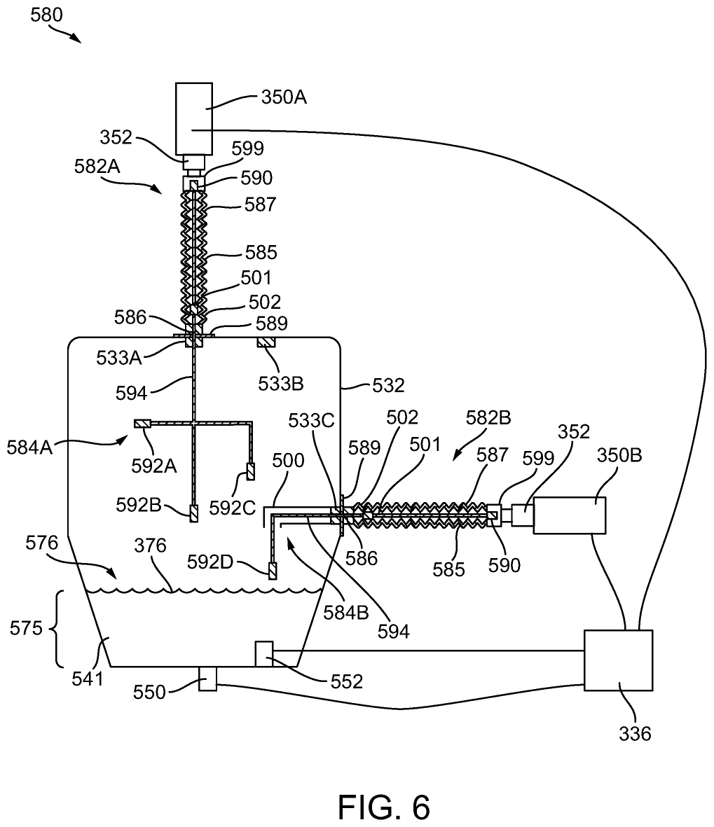

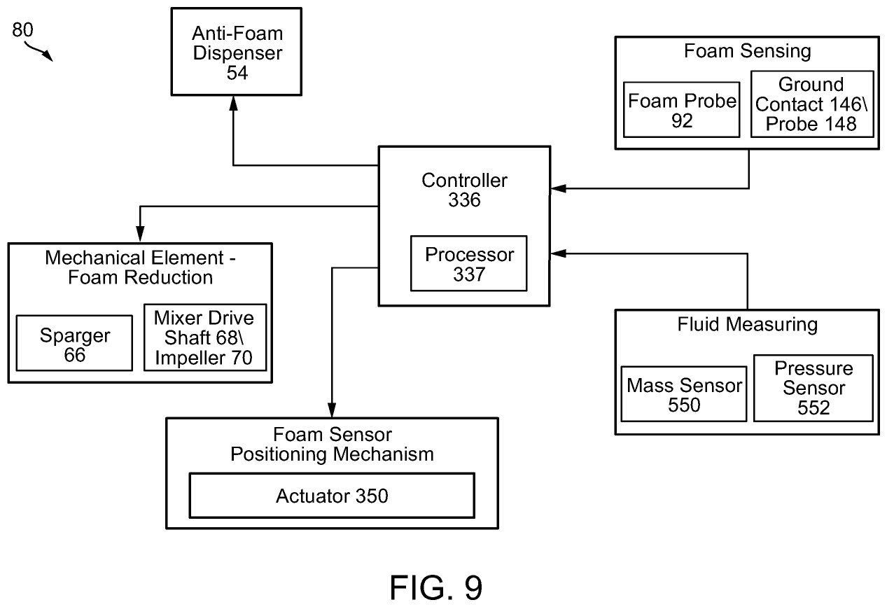

As depicted in FIG. 1, the adjustable foam sensor system 80 is integrated with fluid processing system 10 to sense, detect, measure, reduce and/or eliminate unwanted foam in fluid processing system 10 and particularly on or near the top surface 76 of the fluid 41 in the container 32. The adjustable foam sensor system 80 can include an adjustable foam sensor assembly 82, an anti-foam dispenser 54 and one or more ground assemblies 140, 140B. In some embodiments, the adjustable foam sensor system 80 further includes a controller 336 (e.g., including processor 337, as shown in FIG. 9) and one or more sensors, measuring devices, and mechanical elements of the fluid processing system 10. For example, the adjustable foam sensor system 80 can include at least one fluid measuring device (e.g., mass measuring sensor 550, pressure measuring sensor 552 shown in FIG. 6) that is in communication with the controller 336 and can assist with detecting and/or measuring a change in fluid levels. As used herein, a fluid level can include a fluid depth and/or a distance between a bottom surface of the container and/or fluid and a top surface of the fluid.

In some embodiments, the adjustable foam sensor system 80 can include the controller 336 in communication with at least one mechanical element of the fluid sensing device (e.g., drive shaft 68 and/or impeller of mixer, sparger 66, etc.) that can assist with reducing and/or eliminating foam in the fluid processing system, such as when the controller determines foam is present in the fluid processing system 10. In some embodiments, the controller 336 can also control the adjustable foam sensor assembly 82 to position an associated foam probe in a beneficial position for efficiently and effectively detecting foam. Such beneficial positioning of the foam probe can be based on determined changes in fluid levels. As such, the adjustable foam sensor system 80 can efficiently and effectively position the foam probe and detect foam, including under varying fluid level conditions. The adjustable foam sensor system 80 can reduce and/or eliminate foam in the fluid processing system 10, such as to prevent damage to the fluid processing system 10 and/or to the fluid being processed. Various embodiments of the adjustable foam sensor system are described and captured herein.

Embodiments of the adjustable foam sensor system 80 and component parts, including adjustable foam sensor assembly 82, one or more ground assemblies 140, 140B and an anti-foam dispenser 54, can send signals to, receive signals from and communicate with the controller 336. The controller 336 can include a memory and a central processing unit (CPU). CPU and memory of controller 336 can include a random access memory (RAM), a computer readable medium, flash memory, magnetic disk drive, optical drive, programmable read only memory (PROM), and/or read only memory (ROM) capable of storing and running software and software applications that control and process signals from fluid processing system 10, control the delivery of anti-foam from the anti-foam dispenser 54 and generally control the process, process parameters and fluid processing system 10.

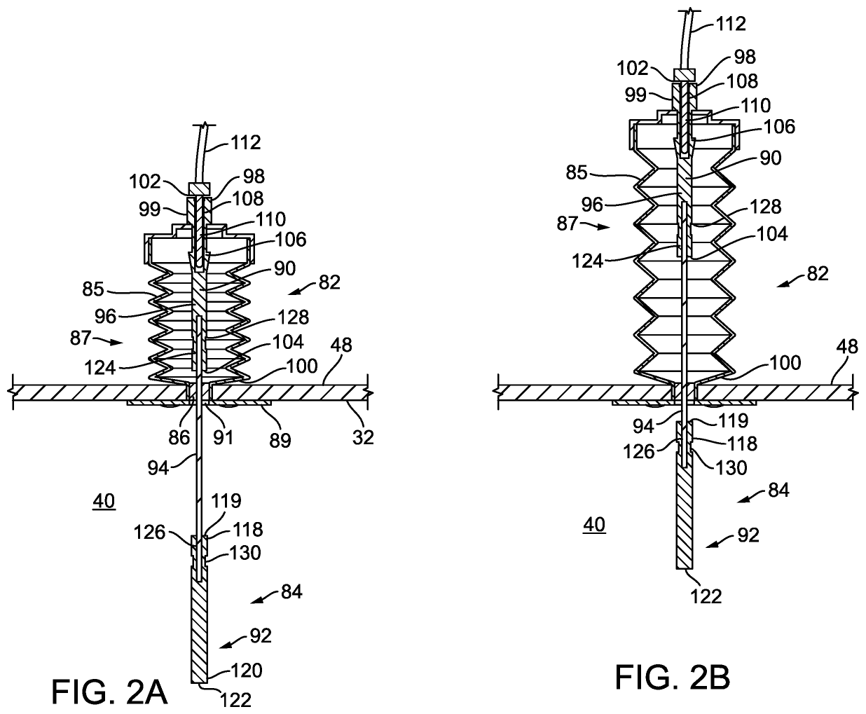

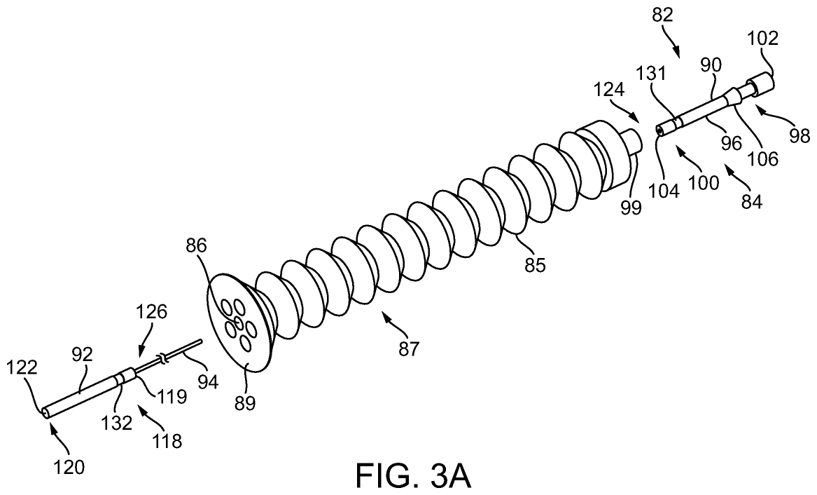

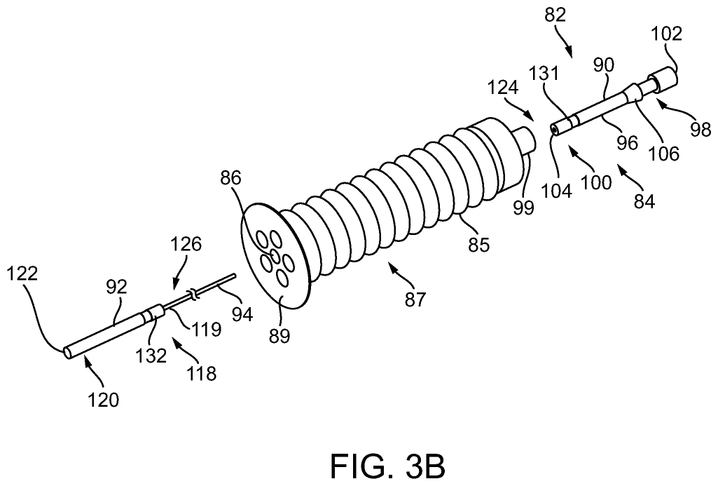

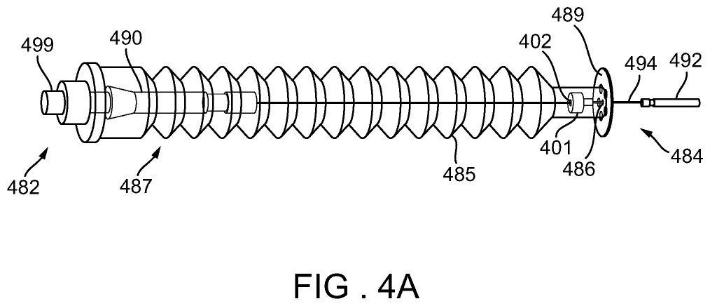

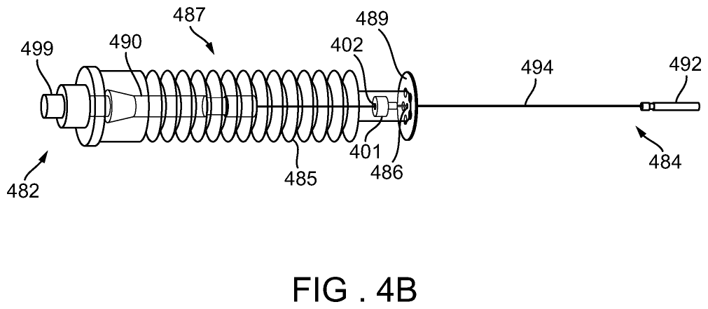

In some embodiments, the adjustable foam sensor assembly 82 can include an adjustable foam sensor 84 with an adjustable sensor housing 87 that facilitates movement of the adjustable foam sensor 84 into a variety of positions within container 32 (e.g., bioprocess container). The components of the adjustable foam sensor 84 can include a base 90, a container interface 89, a foam probe 92, a transition member 94 and a sensor probe guide (shown as 401 in FIGS. 4A-4B). The container interface 89 can attach to, connect and/or otherwise interface with the container 32 to create a hermetic seal with the container 32. For instance, the container interface 89 can be a flange of any shape, such as an annular flange 89 with one or more openings 86 (shown in FIGS. 2A-3B) through which the transition member 94 of the adjustable foam sensor 84 is received and disposed. The transition member 94 can move through (e.g., linearly translate and/or rotationally translate) the opening 86 (shown in FIGS. 3A-3B), such as during use, extension, and/or retraction of the adjustable foam sensor 84. The container interface 89 can connect or otherwise attach to the container 32 at a port formed in the container 32 to create an air-tight and/or liquid-tight seal. In an exemplary embodiment, the container interface 89 can include a ½ inch face port opening. The container interface 89 can also have a larger or smaller port opening depending on the size of the adjustable foam sensor 84.

In exemplary embodiments, the adjustable sensor housing 87 has a housing body 85 and a housing base 99. The housing body 85 can be formed from pliable and flexible bellows that can be compressed (as shown in FIG. 2A) and extended (as shown in FIG. 2B) like a spring. The housing base 99 can be formed from a threaded or unthreaded block of rigid material capable of absorbing forces (e.g., pull and push forces) and transferring those forces to the housing body 85 and housing base 99 of the adjustable foam sensor 84. The housing body 85 attaches to, connects to and/or otherwise interfaces with the container interface 89 at one end and attaches to, connects to and/or otherwise interfaces with the housing base 99 at the opposing end. The housing body 85 and housing base 99 can be formed of the same or different material and can be integrated as a single part or two different parts that are coupled together to form a single part (e.g., the adjustable sensor housing 87).

To actuate, move, extend or retract the adjustable foam sensor 84 relative to the container 32, a force can be applied to an end of the adjustable sensor housing, such as the housing base 99. Such an applied force can cause the adjustable sensor housing 87 to extend or collapse (e.g., compress). For example, the adjustable sensor housing 87 can be shortened, retracted and/or compressed (as shown in FIG. 2A) by applying a push force (e.g., manually by a user and/or by an actuator) to the housing base 99 while retaining the container interface 89 in a fixed or substantially fixed position (e.g., relative to the container 32). Likewise, the adjustable sensor housing 87 can be stretched, lengthened and/or extended (as shown in FIG. 2B) by applying a pull force to the housing base 99 while retaining the container interface 89 in a fixed or substantially fixed position (e.g., relative to the container 32). The container interface 89 of the adjustable foam sensor 84 can be fixed or substantially fixed to the container 32 to facilitate actuation of both the adjustable sensor housing 87 and the adjustable foam sensor 84.

After securing the container interface 89 of the adjustable foam sensor 84 to the container 32, for example, a push force applied to the housing base 99 also causes and applies a push force to the adjustable foam sensor base 90, which in-turn causes the flexible bellows of the housing body 85 to compress and the foam probe 92 and/or a portion of the transition member 94 to extend and/or increasingly extend into the container 32. Likewise, a pull force applied to the housing base 99, for example, causes and applies a pull force to the adjustable foam sensor base 90, which in-turn causes the flexible bellows of the housing body 85 to extend and the foam probe 92 and/or a portion of the transition member 94 to retract towards and/or retract from the container 32. With the use of the exemplary adjustable foam sensors 84 with adjustable sensor housing 87, the foam probe 92 and/or a portion of the transition member 94 can be moved relative to the chamber, such as moved within the chamber including fully and/or partially moving in and out of the container 32. For example, the foam probe 92 can be positioned within the chamber based on fluid 41 levels, such as to allow the foam probe 92 to efficiently and effectively detect foam at a variety of locations within the container 32, including along and/or above the top surface 76 of the fluid 41.

Although depicted in FIG. 1 as mounted to the top end wall 48 of container 32, exemplary adjustable foam sensors 84 can be moved, retracted or extended in and out of the container 32 along any part of the container, such as to the top end wall 48, the sidewall 42 or bottom end wall 50 of the container 32. During use, the container interface 89 may be substantially fixed. Extension of the adjustable foam sensor 84 can include the foam probe 92 fully or partially advancing into the container 32, such as relative to a container wall and/or relative to the adjustable sensor housing 87 of the adjustable foam sensor assembly 82. Additionally, retraction of the adjustable foam sensor 84 can include fully or partly retracting the foam probe 92 relative to the adjustable sensor housing 87, including retracting within and/or out of the container 32.

In some configurations, pressure within an inflated or partially inflated container 32 provides a counterforce and support to the container interface 89 during actuation of the adjustable sensor housing 87 and adjustable foam sensor 84. FIGS. 4A-4B, which are later described in detail, depict an exemplary adjustable foam sensor assembly 482 with the adjustable foam sensor 484 and adjustable foam sensor housing 487 in the retracted and extended positions.

In exemplary embodiments disclosed herein, a push force applied to the housing base 99 (e.g., applied manually or via an actuator, such as actuator 350A of FIG. 6) compresses the flexible bellows of the housing body 85, and a pull force applied to the housing base 99 (e.g., applied manually or via an actuator, such as actuator 350A of FIG. 6) extends the flexible bellows of the housing body 85. Other forces can also be applied to the sensor housing 87, and other housing configurations can also be used to actuate both the adjustable foam sensor housing 87 and adjustable foam sensor 84, such as to position and reposition the foam probe 92 at distinct locations within the fluid processing system 10.

This dual actuated adjustable foam sensor assembly 82 can also be equipped with signal clarifying equipment that reduces and/or prevents false signals and signal fouling. For example, adjustable foam sensors 84 disclosed herein can also include a sensor probe guide with one or more openings or through-holes (shown in FIG. 4A as 401, 402). The transition member 94 of adjustable foam sensor 84 can be fed through or disposed in an opening or through-hole of the sensor probe guide (shown in FIG. 4A as 401, 402) to secure, guide and center (e.g., relative to the adjustable sensor housing 87 and/or relative to the opening 86 of the container interface 89) the transition member 94 and the foam probe 92 during use. During extension and retraction of the adjustable foam sensor 84 and adjustable sensor housing 87, the transition member 94 can move within and/or along the opening in the sensor probe guide (shown in FIG. 4A as 401, 402). The sensor probe guide (shown in FIG. 4A as 401, 402) centers the adjustable foam sensor 84, including the transition member 94 and the foam probe 92, during use so that the transition member 94 or any part of the adjustable foam sensor 84 does not contact other surfaces, including the container interface 89, the adjustable sensor housing 87 or any part of the fluid processing system 10 or container 32. By centering, guiding and precluding contact of the adjustable foam sensor 84, including the transition member 94 and the foam probe 92, with other surfaces, the sensor probe guide (shown in FIG. 4A as 401, 402) prevents false signals and signal fouling caused by unwanted contact with equipment or fluid surfaces. The sensor probe guide (shown in FIG. 4A as 401, 402) can be positioned proximate to and/or coupled, attached or connected to the container interface 89.

The housing base 99 of the adjustable foam sensor housing 87 can include an internal or external threaded portion, a barb fitting or quick connect fitting used to connect to an autoclave kit for sterilization. An internal threaded portion, a barb fitting or quick connect fitting of the housing base 99 can be used to attach to the sensor base 90. Due to its versatility and adjustability, the adjustable foam sensor assembly 82 can be attached and sealed to the container 32 prior to irradiation. Since the foam probe 92 can be adjusted after installation without breaking hermetic seals, the adjustable foam sensor assembly 82 coupled and hermetically sealed to the container 32 as one unit can be irradiated and sterilized together and used without ever decoupling the adjustable foam sensor assembly 82 and container 32 through the entire process. This assures the most sterile use of the adjustable foam sensor assembly 82 within a variety of biological processes and bioproduction equipment 10.

The adjustable foam sensor system 80 can also come equipped with one or more ground assemblies 140, 140B. In general, ground assembly 140 comprises a tube assembly 142 (which can also be referred to herein as a housing), a tube port 33E that couples tube assembly 142 to container 32, a ground contact 146 coupled to an end of tube assembly 142, and a probe 148 that is received within tube assembly 142 and engages with ground contact 146. Ground assembly 140B can couple to port 33F and have similar functionality as ground assembly 140. More detailed descriptions of the components and functions of ground assembly 140, 140B and other ground assemblies are provided in U.S. Pat. No. 9,606,077, issued Mar. 28, 2107, and incorporated herein by specific reference in its entirety.

In exemplary embodiments depicted in FIG. 1, both the probe 148 of ground sensor 140 and the foam probe 92 of the adjustable foam sensor 84 are in electrical communication with the controller 336. For example, the controller 336 can apply an electrical potential or voltage between the ground probe 148 (and thus also ground contact 146) and the adjustable foam sensor 84 during normal operation. During the operation of fluid processing system 10, foam can build on the top surface 76 of fluid 41. The adjustable foam sensor 84 can detect foam that contacts the foam probe sensor 92. Once in contact with the foam, the adjustable foam sensor 84 can send electrical signals (e.g., changes in electrical signals) indicative of the presence, concentration, density and/or volume of foam (e.g., voltage, current or other signals) to the controller 336. Typically, foam builds on the top surface 76 of the fluid 41 so as to contact an outer surface of the foam probe 92 of the adjustable foam sensor 84, which can trigger the transmission of an electrical signal from the adjustable foam sensor 84, through the foam, fluid 41 and ground contact 146/probe 148 and to the controller 336. In some embodiments, the contact between the adjustable foam sensor 84 and foam triggers the transmission of a signal indicative of the concentration, density or volume of foam. The adjustable foam sensor 84 can also continuously detect foam concentration, density or volume in fluid processing system 10 throughout the processing of fluid. In other exemplary embodiments, the controller 336 can automatically trigger detection of foam concentration, density or volume at a location proximate the foam probe 92 by pinging and/or controlling the adjustable foam sensor 84 (e.g., moving the foam probe 92 to more than one location within the chamber 32 to detect presence of foam in the more than one locations).

The electrical signals indicative of foam concentration, density or volume sent from the adjustable foam sensor 84 are received and processed by the controller 336, and the controller 336 can output a human and/or machine-readable magnitude or level of foam (e.g., depth of foam, volume of foam, etc.). Based on the magnitude or level of foam present, the controller 336 can transmit a signal to an anti-foam dispenser 54 that releases a predetermined or preprogrammed quantity, flow rate, density and/or volume of an anti-foaming agent into the fluid processing system 10, including the container 32, based on the concentration, density or volume of foam detected. The predetermined quantity, flow rate, density and/or volume of anti-foaming agent can temporarily or permanently dissipate, diminish or eliminate the foam buildup.

Controller 336 can be programed in a variety of ways to dispense the anti-foaming agent by controlling anti-foam dispenser 54. For example, the anti-foaming agent can be dispensed as a large bolus after which controller 336 waits for a period of time before checking again or pinging for an electrical signal from the adjustable foam sensor 84. Alternatively, the anti-foaming agent can be slowly and continuously released once the signal is detected from the adjustable foam sensor 84 and then stopped once controller 336 can no longer detect the electrical signal indicative of foam levels. In some embodiments, a volume of anti-foaming agent for reducing and/or eliminating foam can be determined based on a measured thickness and/or volume of the foam. As such, the determined volume of anti-foaming agent can be delivered to the container 32 for sufficiently reducing and/or eliminating foam in the container 32 without adding excess anti-foaming agent to the fluid 41. Other control methods and feedback loops can also be used.

By continuously monitoring the presence of foam and/or foam levels within the container 32 using the adjustable foam sensor system 80, the amount of foam within the container 32 can be maintained sufficiently low, and the risk of the foam effects can be reduced (e.g., system 80 prevents foam from clogging gas filter 58). In addition, foam sensor system 80 can dispense the amount of anti-foaming agent needed to eliminate the foam or maintain the foam at a desired level. As a result, less anti-foaming agent can be added to the fluid, and thus, less anti-foaming agent needs to be removed from the fluid and/or culture.

Controller 336 can also be programed to automatically actuate, extend and retract the adjustable foam sensor 84 and/or adjustable sensor housing 87, such as via an actuator coupled directly or indirectly to the adjustable foam sensor 84 and the adjustable sensor housing 87 in order to apply a force to the adjustable foam sensor 84 and/or adjustable sensor housing 87. As liquid levels rise or fall within bioproduction equipment 10, controller 336 can appropriately actuate the adjustable foam sensor 84 and adjustable sensor housing 87 to extend, retract and/or position the foam probe 92 above or proximate to the fluid 41 surface in the container 32.

One of the challenges of foam is the relatively sticky properties of foam that can cause the foam to adhere to both the interior surface of container 32 and to components of the adjustable foam sensor 84. As a result of the gas flowing through gap 78 and the humid vapor within gap 78 that can carry small particles of foam, a thin layer of foam can build up on interior surface 38 of container 32 within gap 78 and on the exposed portion of adjustable foam sensor 84 within gap 78. In addition, the foam does not generally build up as an even layer on top surface 76 of fluid 41 but typically builds up in clumps. The clumps may obtain a height that extends up to transition member 94 (shown in FIG. 2) before the foam first encounters the adjustable foam sensor 84. These clumps can cause buildup of foam on the interior surface of container 32 within gap 78 and on the exposed portion of adjustable foam sensor 84 within gap 78. If a continuous layer of foam is formed on interior surface 38 of container 32 from the top surface 76 of fluid 41 to the adjustable foam sensor 84, an electrical signal (“false signal”) can pass between foam sensor 84, through ground contact 146, foam and/or fluid 41 and to controller 336. This false signal can give a false reading to the controller 336 regarding the concentration, density or volume of foam in the fluid processing system 10. For example, this type of signal fouling can falsely indicate that the foam layer on fluid 41 has reached the foam probe 92, and thus, trigger the dispensing of anti-foaming agent into fluid 41 when no anti-foaming agent may be needed. Furthermore, foam layers and build up on container 32, can cause a false reading that continues even after the anti-foaming agent is added, thereby resulting in continued or repeated unwanted dispensing of anti-foaming agent into fluid 41.

As previously discussed with respect to the sensor probe guide (shown in FIG. 4A as 401), exemplary adjustable foam sensor assemblies 82 can be equipped with a variety of signal clarifying equipment that reduces and/or prevents false signals and signal fouling. For example, the adjustable foam sensor 84 can be specifically designed with transition member 94 having a smaller diameter than foam probe 92 to help differentiate between a true signal where the signal is produced as a result of foam building up on top surface 76 of fluid 41. In these embodiments, the transition member 94 accounts for foam build-up and prevents false signals where the signal is produced as a result of a thin film of foam coating the interior surface of container 32 that extends between adjustable foam sensor 84 and fluid 41. Specifically, electrical conductance is in part related to the surface area of an electrical contact and the volume of the material through which the electrical current passes. Accordingly, the electrical current of the true signal will always be greater than the electrical current of the false signal. This is true because the volume of foam through which the true electrical signal passes between adjustable foam sensor 84 and fluid 41 is larger than the volume of foam through which the false electrical signal passes on interior surface 38 of container 32 between foam sensor 84 and fluid 41. Furthermore, in embodiments where foam probe 92 has a larger diameter than transition member 94, foam probe 92 will have more surface area contacting the foam on top of fluid 41 than transition member 94 will have contacting the thin film of foam on the surface of container 32.

Accordingly, controller 336 can be programmed so that when electrical signals from the adjustable foam sensor system 80 are below a predetermined value, it is assumed to be a false signal and no anti-foaming agent is released. However, when the signal exceeds the predetermined value, it is designated as a true and accurate signal, and the anti-foaming agent is released as discussed above. The predetermined value on which to determine a true or false signal can be the measured electrical signal strength or conductivity. For example, in one embodiment, only signals having a conductivity of greater than 20μ Siemens and more commonly greater than 30μ Siemens or 40μ Siemens will be determined to be a true signal. It is appreciated that the predetermined conductivity value can be set over a wide range depending on factors such as, the amount of voltage applied between foam probe 92 and foam ground 146, the relative diameters between transition member 94 and foam probe 92, the materials used for the probes and other factors. In other embodiments, the predetermined value can be set at any value between 20μ Siemens and 50μ Siemens. Other values can also be used. Likewise, other measurements, such as current, can also be used as the predetermined value.

To help differentiate between the true signal and the false signal, foam probe 92 can have a diameter normal to the longitudinal length thereof that is at least 3, 4, 5, 6, 8, or 10 times larger than a diameter of transition member 94 disposed within chamber 40 of container 32 as measured normal to the longitudinal length thereof. Expressed in other terms, a diameter of transition member 94 can be at most ⅓, ¼, ⅕, ⅙, ⅛, or 1/10 of a diameter of foam probe 92. Because diameters can change along the length of foam probe 92 and transition member 94, the above measured and compared diameters for foam probe 92 and transition member 94 can be selected as a maximum diameter, minimum diameter, average diameter over the length thereof or a diameter at any location on or over at least a portion of foam contact 92 and transition member 94. Other ratios can also be used. The diameter of the foam probe 92 is typically greater than 2 mm, 3 mm, 5 mm, 7 mm or 10 mm or in a range between 2 mm an 10 mm while the diameter of transition member 94 is typically less than 2.5 mm, 2 mm, 1.5 mm, 1 mm, 0.75 mm or 0.5 mm or in a range between 2.5 mm and 0.5 mm. These listed example diameters can be a maximum diameter, minimum diameter, average diameter over the length or a diameter at any location on or over at least a portion of foam probe 92 or transition member 94. Other dimensions can also be used. It is noted that the term “diameter” as used herein refers to a straight line or the length of such line passing from side to side of the corresponding structure, through its center, and is not intended to limit the structure to a circular or any other defined shape.

FIGS. 2A-3B depict an exemplary adjustable foam sensor assembly 82. The adjustable foam sensor assembly 82 includes, in part, an adjustable foam sensor 84, an adjustable sensor housing 87, a foam probe 92, a sensor base 90 and a transition member 94 that extends between the foam probe 92 and the sensor base 90. The adjustable sensor housing 87 includes a housing base 99 and housing body 85. FIG. 2A depicts the adjustable sensor housing 87 in a compressed configuration and FIG. 2B depicts the adjustable sensor housing 87 in an extended configuration according to embodiments of the present subject matter. As shown in FIGS. 2A and 2B, the foam probe 92 is positioned further away from the adjustable sensor housing 87 and wall of the container 32 when the adjustable sensor housing 87 is in the compressed configuration compared to the extended configuration.

The sensor base 90 includes an elongated body 96 that is typically cylindrical and extends between a first end 98 and an opposing second end 100. The sensor base 90 can be received within opening 86 of container interface 89, such as during assembly (e.g., during use) and/or manufacturing. First end 98 terminates at a first end face 102 while second end 100 terminates at a second end face 104. An annular barb 106 can encircle and radially outwardly project from body 96 at a location between first end 98 and second end 100. In exemplary embodiments, barb 106 is disposed at or towards first end 98. As needed, a tie, crimp, or other clamp can encircle and form a constricting force on the exterior surface of stem 34 adjacent the barb 106 (e.g., at or towards first end 98) to enhance the seal against barb 106. The sensor base 90 can be made of metal or other electrically conductive material. In exemplary embodiments, sensor base 90 is stainless steel. However, the sensor base 90 can be made of other metals, combinations of metals, alloys and other material. Furthermore, although sensor base 90 is shown as being formed as a single integral member, sensor base 90 can also be formed from multiple members connected together and from a plurality of stands of wires bundled, woven, or otherwise secured together, such as a cable. As needed, barb 106 can be replaced with other structure that forms a liquid tight seal with the housing base 99. In exemplary embodiments, the adjustable sensor housing 87 can be over-molded onto the sensor base 90 or otherwise secured or fastened thereto so that a liquid tight seal is formed therebetween.

As also depicted in FIGS. 2A and 2B, a socket 108 is formed on first end face 102 and longitudinally projects into body 96. An electrical plug 110 having electrical wiring 112 is configured to be received within socket 108, such as in a friction fit connection, so that a positive electrical contact is made between plug 110 and sensor base 90. In exemplary embodiments, electrical wiring 112 can be permanently secured to body 96 such as through soldering, crimping, or other electrical connections. Electrical wiring 112 can electrically connect to controller 336 (shown in FIG. 1) to facilitate transmission and communication of signals.

Referring to FIGS. 2A-3B, the foam probe 92 can be elongated and extend between a first end 118 that terminates at a first end face 119 and an opposing second end 120 that terminates at a second end face 122. In exemplary embodiments, foam probe 92 has a length between end faces 119 and 122 in a range between approximately 0.5 cm and 15 cm, such as between approximately 1 cm and 8 cm or approximately 2 cm and 6 cm. Other dimensions can also be used. Although not required, in the depicted embodiments, foam probe 92 has a substantially cylindrical configuration extending along the length thereof. In alternative embodiments, foam probe 92 can have alternative transverse cross-sectional configurations such as polygonal, elliptical, irregular, or the like. The foam probe 92 is also made of a metal or other electrically conductive material and is typically made of stainless steel. However, other metals can also be used. Furthermore, although the foam probe 92 is shown as being formed as a single integral member, it can also be formed from multiple members connected together and from a plurality of stands of wires bundled, woven, or otherwise secured together, such as a cable.

In contrast to sensor base 90 and foam probe 92, which are typically made of a relatively rigid metal, in exemplary embodiments, transition member 94 can be made from a highly resiliently flexible wire that is comprised of metal or other electrically conductive material. For example, transition member 94 can be made from a memory metal. Examples of memory metals include, but are not limited to, nickel-titanium alloys that commonly sold under the name nitinol and copper-aluminum-nickel alloys. Transition member 94 can be made from a material that enables it to be bent over an angle of at least 90° and more commonly at least 180°, 270° or at least 360° without plastic deformation. In exemplary embodiments, transition member 94 can be made of a wire that bends with plastic deformation and the sensor base 90 and/or foam probe 92 can be made or similar or different material. In other embodiments, the transition member 94 can be a relatively small diameter shaft rather than a wire. Furthermore, although transition member 94 is shown as being formed as a single integral member, transition member 94 can also be formed from multiple members connected together and from a plurality of strands of wires bundled, woven, or otherwise secured together, such as a cable. In addition, transition member 94 can be formed as a single unity member with sensor base 90 and/or foam probe 92. For example, sensor base 90, transition member 94, and foam probe 92 can be molded, stamped, or cut so that they form one continuous member as opposed to two or more separate members that are secured together. Transition member 94 is typically made of a different material than sensor base 90 or foam probe 92. Sensor base 90 and foam probe 92 are typically made from the same material but it is not required. In some embodiments, the transition member 94 can be flexible and the foam probe 92 can be formed to float along the top surface of the fluid and thus not become submerged in the fluid 41, such as when the fluid level rises at least up to a position where the foam probe 92 is positioned. For example, the foam probe 92 can be formed such that at least part of the foam probe 92 has a density that is less than the fluid and/or water. As such, at least a part of the foam probe 92 can maintain above the top surface of the fluid 41 to detect the presence of foam in the container 32.

In exemplary embodiments, to attach transition member 94 to sensor base 90 and foam probe 92, a socket 128 (shown in FIGS. 2A and 2B) is formed on second end face 104 of sensor base 90, and a socket 130 is formed on first end face 119 of foam contact 92. First end 124 of transition member 94 is received within a first socket 128 while first end 126 of transition member 94 is received within a second socket 130. A crimp force can then be applied around a portion of sensor base 90 and foam probe 92 encircling transition member 94 so that the opposing ends of transition member 94 are held by crimp connection within sensor base 90 and foam probe 92. The crimping force can produce a recessed crimp groove 131 (shown in FIGS. 3A-3B) on sensor base 90 and a crimp groove 132 (shown in FIGS. 3A-3B) on foam probe 92. Other methods of attachment can also be used. The exposed portion of transition member 94 can have a length in a range between 2 cm and 15 cm and more commonly between 3 cm and 10 cm or 4 cm and 8 cm. Other dimensions can also be used.

By making transition member 94 out of a resiliently flexible wire, container system 30 can be folded or rolled up for storage, transport, and/or sterilization even after the adjustable foam sensor 84 has been attached without risk of damage to the adjustable foam sensor 84 or to container 32. For example, the transition member 94 bends when container system 30 is folded or rolled up so that the adjustable foam sensor 84 does not break or puncture container 32. When container 32 is unfolded and inflated, transition member 94 resiliently returns to its original desired configuration. Transition member 94 is also shown as having a smaller diameter than foam probe 92.

The exemplary adjustable foam sensor assemblies 82 can be assembled as depicted in FIGS. 2A and 2B. The sensor base 90 can be received within opening 86 and form a liquid tight seal with housing base 99. Second end face 104 of sensor base 90 interfaces with transition member 94 at first end 124 of sensor base 90 while first end 126 of transition member 94 is disposed within the compartment 40 of a container 32 (shown in FIG. 1). Foam probe 92 is disposed completely within compartment 40 of container 32 and is typically positioned so that end face 122 is adjusted to any distance from top end wall 48 of container 32 during operation of the fluid processing system 10. Other configuration can also be implemented depending on the biological process and application. For example, in exemplary embodiments, the adjustable foam sensor assembly 82 can also be mounted on the sidewall 42 of a container 32 and/or at first end 44 of the container (shown in FIG. 1). When mounting the adjustable foam sensor assembly 82 to the sidewall 42 of container 32, the foam probe 92 can be positioned any distance from the sidewall 42 (shown in FIG. 1). Regardless of mounting location, the gas pressure within the compartment 40 can support and maintain the bioprocess container 32 in an inflated position and/or support the adjustable foam sensor assembly 82 in a mounted position on the bioprocess container 32.

As depicted in FIGS. 2A and 2B, in the assembled state a cavity 91 (comprising a portion of opening 86) is formed in container interface flange 89 to approximately second end face 104 of sensor base 90. Transition member 94 centrally extends through cavity 91 with an annular gap formed to accept transition member 94. Because the system can be pressurized as a result of the inflow of sparging gas, foam will typically not enter or build up within cavity 91. However, foam can bridge between transition member 94 or container interface flange 89 and sidewall of cavity 91. The bridging typically occurs as a result of a clump of foam contacting and adhering to transition member 94, as a result of foam collecting within compartment 40 of container 32 and remaining on transition member 94 even when the remainder of the foam is dissipated as a result of the addition of an anti-foaming agent. The false signal or signal fouling can be produced as a result of the foam bridge contacting the foam build-up on interior surface 38 of container 32, thereby completing the circuit to ground contact 146 (shown in FIG. 1), as discussed above.

To help eliminate or minimize the formation of a foam bridge between transition member 94, tubular stem 88 and container interface flange 89 (and thereby minimize any false signal), the diameter of opening 86/cavity 91 within adjustable sensor housing 87 can be increased relative to the diameter of transition member 94. For example, while the diameter of transition member 94 is typically in the values as discussed above, the inside diameter of opening 86/cavity 91 encircling transition member 94 is typically greater than approximately 5 mm, 10 mm, 15 mm, 20 mm, 30 mm, 40 mm or 50 mm. Other dimensions can also be used. In general, the larger the diameter, the lower the probability that a foam bridge can be formed and maintained between transition member 94 and flange container interface 89.

In exemplary embodiments, the adjustable foam sensor assembly 82 can be mounted or disposed on sidewall 42 of container 32 and within head space 78 (shown in FIG. 1). In this embodiment, the adjustable sensor housing 87 can be angled downward relative to the horizontal axis so that condensate formed within cavity 91 freely flows out of cavity 91 and into chamber 40 of container 32. This configuration helps to prevent the condensate from collecting in cavity 91 which can cause a false signal or signal fouling. In exemplary embodiments, the adjustable sensor housing 87 of the adjustable foam sensor 84 can be positioned so that a longitudinal axis centrally extending through opening 86 or cavity 91 of adjustable sensor housing 87 is disposed at a downward angle relative to the horizontal in a range between about 10° to about 70°, such as between about 30° to about 45°. Other angles can also be used.

In exemplary embodiments, it is also envisioned that adjustable foam sensor 84 does not include a transition member 94. For example, the foam probe 92 can extend down to body 96 and have a constant diameter along the length thereof. A coating or insulative material can coat the center of the foam probe 92 to prevent foam from sticking thereto and to reduce signal fouling.

As depicted in FIGS. 3A-3B, the adjustable sensor housing 87 can include a housing body 85 and a housing base 99. The housing body 85 can be formed from pliable and flexible bellows that can be compressed and extended like a spring. The housing base 99 can include or be formed from a threaded or unthreaded block of rigid material capable of absorbing forces (e.g., pull and push forces) and transferring those forces to the housing body 85. The housing base 99 of the adjustable sensor housing 87 can include an internal or external threaded portion, a barb fitting or quick connect fitting used to connect to an autoclave kit for sterilization. An internal threaded portion, a barb fitting or quick connect fitting of the housing base 99 can be used to attach to the sensor base 90. The housing body 85 attaches to, connects to and/or otherwise interfaces with the container interface 89 at one end and attaches to, connects to and/or otherwise interfaces with the sensor base 90 of the of the adjustable foam sensor 84 at the other end. In exemplary embodiments, the container interface 89 can include a ½ inch barbed faced port that attaches to the housing body 85, and the housing base 99 can include a barbed, threaded, and/or quick connect adaptor that can connect to the sensor base 90.

To actuate, move, extend or retract the adjustable foam sensor 84 relative to a wall of a container 32 (shown in FIG. 1), a force can be applied to the housing base 99, causing the adjustable sensor housing 87 to extend or compress. As shown in FIGS. 2B and 3A, the adjustable sensor housing 87 can be stretched, lengthened and/or extended by applying a pull force to the housing base 99 while retaining the container interface 89 in a fixed or substantially fixed position. In this configuration, when the adjustable sensor housing 87 is extended, the adjustable foam sensor 84 is fully or partially retracted from within the container 32. Likewise, as shown in FIGS. 2A and 3B, the adjustable sensor housing 87 can be shortened, retracted and/or compressed by applying a push force to the housing base 99 while retaining the container interface 89 in a fixed or substantially fixed position, such as in a fixed or substantially fixed position along the container 32, as shown in FIG. 1. In this configuration, when the adjustable sensor housing 87 is compressed, the adjustable foam sensor 84 is advanced away from the adjustable sensor housing 87 and extended into the container 32 (shown in FIG. 2A). The container interface 89 of the adjustable foam sensor 84 can be a fixed or substantially fixed to the container 32 (shown in FIG. 1) or a port thereof to facilitate actuation of both the adjustable sensor housing 87 and the adjustable foam sensor 84. In some embodiments, the housing base 99 can be coupled to an actuator (such as actuator 350 coupled to housing base 599 in FIG. 6) to allow the actuator to apply the pushing and pulling forces to the housing base 99 and thereby transition the adjustable sensor housing 87 into the compressed and extended configurations, respectively.

In other exemplary embodiments, the adjustable sensor housing 87 and adjustable foam sensor 84 can be stretched, lengthened and/or extended by applying a pull, push or other force, and can be shortened, retracted and/or compressed by applying a push, pull or other force. After securing the container interface 89 of the adjustable foam sensor 84 to the container 32 (shown in FIG. 1), a push force applied to the housing base 99 can also cause and apply a push force to the adjustable foam sensor base 90, which in-turn decreases a distance or length between the foam sensor base 90 and the container interface 89. Such decrease in distance can cause the flexible bellows of the housing body 85 to compress and the foam probe 92 and/or a portion of the transition member 94 to extend and/or advance into the container 32 (shown in FIGS. 1 and 2A). Likewise, a pull force applied to the housing base 99 can also cause and apply a pull force to the adjustable foam sensor base 90, which in-turn increases a distance or length between the foam sensor base 90 and the container interface 89. Such increase in distance can cause the flexible bellows of the housing body 85 to extend and the foam probe 92 and/or a portion of the transition member 94 to retract from the container 32 (shown in FIGS. 1 and 2B). With the use of the exemplary adjustable foam sensors 84 that also have an adjustable sensor housing 87, the foam probe 92 and/or a portion of the transition member 94 can be freely extended and retracted in and out of the container 32 to different fluid levels, heights and locations to detect foam at a variety of locations within the container 32. In exemplary embodiments disclosed herein, a push force applied to the housing base 99 compresses the flexible bellows of the housing body 85, and a pull force applied to the housing base 99 extends the flexible bellows of the housing body 85. Other forces can also be applied to the adjustable sensor housing 87 and other housing configurations can also be used to actuate both the adjustable foam sensor housing 87 and adjustable foam sensor 84 and position the foam probe 92 at distinct locations within bioproduction equipment. In some embodiments, an actuator is incorporated in order to provide the push and/or pull forces.

FIGS. 4A-4B depict an exemplary adjustable foam sensor assembly 482 with adjustable foam sensor 484 and adjustable sensor housing 487 in the extended and retracted states, respectively. The exemplary adjustable foam sensor assembly 484 can include an adjustable foam sensor 484 with an adjustable sensor housing 487 that facilitates the free movement of the adjustable foam sensor 484 into a variety of positions within a container 32 (shown in FIG. 1) or another container. The adjustable foam sensor 484 includes a sensor base 490, a container interface 489, a foam probe 492, a transition member 494 and a sensor probe guide 401. The container interface 89 can attach to, connect and/or otherwise interface with the container 32 (shown in FIG. 1) to create a hermetic seal between the container interface 489 and container 32. The container interface 489 can be a flange of any shape, including an annular flange 489 with one or more openings 486 through which the transition member 494 of the adjustable foam sensor 484 is received and disposed. The transition member 494 can freely move through, in and out of the opening 486 during use, extension, and retraction of the adjustable foam sensor 484. The container interface 489 can connect or otherwise attach to the container 32 at a port formed in the container 32 (shown in FIG. 1) to create an air-tight and/or liquid-tight seal. In an exemplary embodiment, the container interface 489 can include a ½ inch face port opening 486 or other size opening (e.g. up to 2 inches).

In exemplary embodiments, the adjustable sensor housing 487 can include a housing body 485 and a housing base 499. The housing body 485 of the adjustable sensor housing 487 can be formed from pliable and flexible bellows that can be compressed and extended like a spring. The housing base 499 can be formed from one or more rigid materials capable of absorbing forces (e.g., pull and push forces) and transferring those forces to the housing body 485 of the adjustable sensor housing 487. One end of the housing body 485 attaches to, connects to and/or otherwise interfaces with the container interface 489, and the other end of the housing body 485 connects to and/or otherwise interfaces with housing base 499. In exemplary embodiments, the housing base 499 can be attached to the sensor base 490 by barbed, threaded, quick connect adaptor or weld that can form an aseptic seal between the two parts.

To actuate, move, extend or retract the adjustable foam sensor 84 in and out of the container 32 (shown in FIG. 1), a force can be applied to the housing base 499, causing the adjustable sensor housing 487 to extend or compress. For example, the adjustable sensor housing 487 can be shortened, retracted and/or compressed by applying a push force (e.g., by an actuator) to the housing base 499 while retaining the container interface 489 in a fixed or substantially fixed position. Likewise, the adjustable sensor housing 487 can be stretched, lengthened and/or extended by applying a pull force to the housing base 499 (e.g., by an actuator) while retaining the container interface 489 in a fixed or substantially fixed position. The container interface 489 of the adjustable foam sensor 484 can be a fixed or substantially fixed to the container 32 (shown in FIG. 1) to facilitate actuation of both the adjustable sensor housing 487 and the adjustable foam sensor 484.

Other forces can also be applied to the adjustable sensor housing 487 and other housing configurations can also be used to actuate both the adjustable foam sensor housing 487 and adjustable foam sensor 484 and position the foam probe 492 at distinct locations within fluid processing equipment.

During use and to move the adjustable foam sensor 484 in and out of the container 32 (shown in FIG. 1), a force can be applied to the adjustable sensor housing 487 to extend and compress the housing 487. For example, after securing the container interface 489 of the adjustable foam sensor 484 to the container 32 (shown in FIG. 1), a push force applied to the housing base 499 also causes and applies a push force to the adjustable foam sensor base 490, which in-turn causes the flexible bellows of the housing body 485 to compress and the foam probe 492 and/or a portion of the transition member 494 to extend and/or advance into the container 32 (shown in FIGS. 2A and 4B). Likewise, a pull force applied to the housing base 499 also causes and applies a pull force to the adjustable foam sensor base 490, which in-turn causes the flexible bellows of the housing body 485 to extend and the foam probe 492 and/or a portion of the transition member 494 to fully or partly retract from the bioprocess container 32 (shown in FIGS. 2B and 4A). The foam probe 492 and/or a portion of the transition member 494 can be partly and/or completely extended and retracted in and out of the container 32, such as in order to adapt to different fluid levels, heights and positions and detect foam at a variety of locations within the container.

FIG. 4A depicts the adjustable sensor housing 487 in the extended state and the adjustable foam sensor 484 in the retracted state. FIG. 4B depicts the adjustable sensor housing 487 in the retracted state and the adjustable foam sensor 484 in the extended state. With this configuration, the adjustable sensor housing 487 is in the extended or expanded state while the adjustable foam sensor 484, including the foam probe 492 and/or a portion of the transition member 494, is in the retracted state (e.g., a shorter length of the transition member 494 and/or foam probe 492 extends into the container 32 compared to the extended state). Likewise, the adjustable sensor housing 487 is in the retracted or compressed state while the adjustable foam sensor 484, including the foam probe 492 and/or a portion of the transition member 494, is in the extended state (e.g., a longer length of the transition member 494 and/or foam probe 492 extends into the container 32 compared to the retracted state).

Exemplary adjustable foam sensors 484 can be moved, retracted or extended partly and/or completely in and out of the container 32 (shown in FIG. 1) while one or more adjustable foam sensors 484 are mounted to the top end wall 48, the sidewall 42 or bottom end wall 50 of the container 32 (shown in FIG. 1). During retraction and extension of the adjustable foam sensor 484 relative to the container, the container interface 489 may be substantially fixed or may move at least to some degree due to the force exerted to the housing base 499. In some configurations, pressure within an inflated or partially inflated container provides a counterforce and support to the container interface 489 during actuation of the adjustable sensor housing 497 and adjustable foam sensor 484. A support structure can also be incorporated with the sensor housing body 485 and base 499 that supports and retains the adjustable foam sensor assembly 482 against the container 32 (shown in FIG. 1). This helps assure that the container interface 489 remains fixed or substantially fixed against the container 32 (shown in FIG. 1), while actuating (e.g., retracting or extending) the adjustable sensor housing 487 and adjustable foam sensor 484.

In exemplary embodiments disclosed herein, a push force applied to the housing base 499 compresses the flexible bellows of the housing body 485, and a pull force applied to the housing base 499 extends the flexible bellows of the housing body 485. Other forces can also be applied to the sensor housing 487 and other housing configurations can also be used to actuate both the adjustable foam sensor housing 487 and adjustable foam sensor 484 and position the foam probe 492 at distinct locations within fluid production equipment.

This dual actuated adjustable foam sensor assembly 482 can also be equipped with signal clarifying equipment that reduces and/or prevents false signals and signal fouling. For example, adjustable foam sensors 484 can also include a sensor probe guide 401 with one or more openings or through-holes 402. The transition member 494 of adjustable foam sensor 84 can be fed through or disposed in an opening or through-hole 402 of the sensor probe guide 401 to secure, guide and center the transition member 494 and the foam probe 492 during use. During extension and retraction of the adjustable foam sensor 484 and adjustable sensor housing 487, the transition member 494 can move through, in and out of the opening 402 in the sensor probe guide 401. The sensor probe guide 401 centers the adjustable foam sensor 484, including the transition member 494 and the foam probe 492, during use so that the transition member 494 or any part of the adjustable foam sensor 484 does not contact other surfaces, including the container interface 489, the adjustable sensor housing 487 or any part of the bioproduction equipment or container. By centering, guiding and precluding contact of the adjustable foam sensor 484, including the transition member 494 and the foam probe 492, with other surfaces, the sensor probe guide 401 prevents false signals and signal fouling caused by unwanted contact with equipment or fluid surfaces. The sensor probe guide 401 can be positioned proximate to and/or coupled, attached or connected to the container interface 489.

In exemplary embodiments, the sensor probe guide 401 is recessed or positioned ¼ inch or more from the container 32 (shown in FIG. 1) or container interface 489 to prevent bridging, which occurs when process fluids bridge gaps between sensor components and other surfaces. In other embodiments, the sensor probe guide 401 can be wedged or press fit with the container interface 489.

The housing base 499 of the adjustable foam sensor housing 487 can include a threaded portion, quick-connect fitting barb or other fitting to connect to an autoclave kit for sterilization, but it is not necessary. Due to its versatility and adjustability, the adjustable foam sensor assembly 482 can be attached and sealed to the container 32 (shown in FIG. 1) prior to irradiation. Since the foam probe 492 can be adjusted after installation without breaking hermetic seals, the adjustable foam sensor assembly 482 coupled and hermetically sealed to the container 32 (shown in FIG. 1) as one unit can be irradiated and sterilized together and used without ever decoupling the adjustable foam sensor assembly 482 and container 432 through the entire process. This assures the most sterile use of the adjustable foam sensor assembly 482 within a variety of biological processes and bioproduction equipment.

The exemplary adjustable sensor housings 487 disclosed herein are not necessarily formed from flexible bellows. The exemplary adjustable sensor housings 487 disclosed herein are simply capable of being extended and/or compressed, such as in response to a force, and different configurations and spring type designs can be used to facilitate the extension or compression of the exemplary adjustable sensor housings 487. As the adjustable sensor housing 487 is extended in response to a force, adjustable foam sensor 484 is retracted, and as exemplary adjustable sensor housing 487 is compressed, adjustable foam sensor 484 is extended. In this way, the adjustable foam sensor 484 can be extended and retracted into and out of containers to detect foam at a variety of locations within fluid processing equipment. The extension and retraction of the exemplary adjustable sensor housing 487 and adjustable foam sensor 484 can be performed manually or automatically with the use of actuation equipment and controller 336 (shown in FIG. 1).

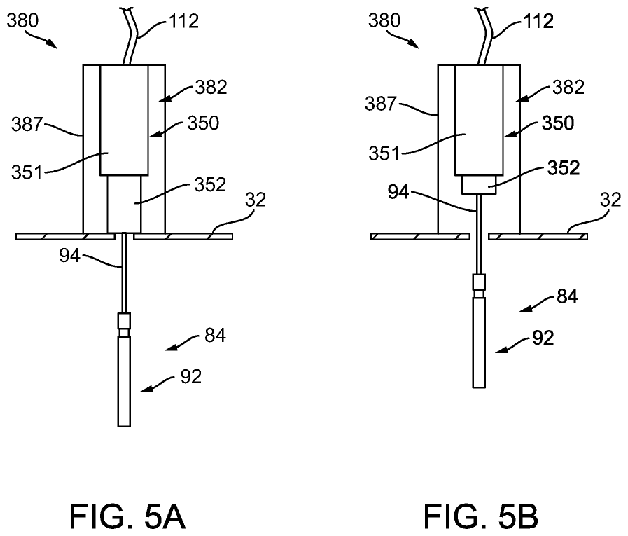

FIGS. 5A and 5B illustrate embodiments of the adjustable foam sensor system 380 including an adjustable foam sensor assembly 382, which includes an adjustable foam sensor 84 and adjustable sensor housing 387. The features and functions of the adjustable foam sensor system 380, including the adjustable foam sensor assembly 382, the foam sensor 84, and the adjustable sensor housing 387, can include the same or similar features and functions as described above with respect to FIGS. 1-4B. For example, the adjustable sensor housing 387 can be securely and hermetically coupled to a wall of the container 32 (shown in FIG. 1), as well as include a foam sensor positioning mechanism that assists with positioning the foam probe 92 in more than one location relative to the wall of the container 32 and/or the adjustable sensor housing 387. As shown in FIGS. 5A and 5B, the foam sensor positioning mechanism can include an actuator 350 that is coupled directly or indirectly to the adjustable foam sensor 84. The actuator 350 can be coupled to the adjustable foam sensor 84 to allow the actuator to move the adjustable foam sensor 84, such as move the foam probe 92 from a first position to a second position within the container 32. The actuator 350 can be coupled to the adjustable sensor housing 387 and/or the actuator 350 and the adjustable sensor housing 387 can be the same unit, such the actuator 350 can be secured to the wall of the container 32.

In some embodiments, the actuator 350 can be a linear actuator, as shown in FIGS. 5A and 5B, such that the adjustable foam sensor 84 is caused to linearly translate (e.g., along a longitudinal axis of the transition member 94 and/or foam probe 92) such that when the actuator 350 is in the extended position, as shown in FIG. 5A, the adjustable foam sensor 84 can position the foam probe 92 a distance from the adjustable sensor housing 87 that is greater compared to when the actuator 350 is in the collapsed configuration, as shown in FIG. 5B. As such, the actuator can transition between at least an extended configuration and a collapsed configuration in order to move the foam probe 92 within the chamber 32.

In some embodiments, the actuator includes a stationary member 351 and a translating member 352 that moves and/or linearly translates relative to the stationary member 351. For example, the translating member 352 can have a sliding engagement with the stationary member 351 and can form a variety of extended and/or collapsed configurations by adjusting a distance the translating member 352 moves relative to the stationary member 351. For example, the translating member 352 can move in increasing and/or decreasing segmented distances relative to the stationary member 351, such as in order to partly extend and/or retract the foam probe 92 within the chamber 32.

As shown in FIGS. 5A and 5B, the actuator 350 can be in communication with wiring 112 that can electrically connect the actuator 350 to controller 336 (shown in FIG. 1) to facilitate transmission and communication of signals. For example, the controller 336 can control when, how far, and in what direction the translating member 352 moves relative to the stationary member 351 to thereby control positioning of the foam probe 92 within the container 32. The actuator 350 can include any number of a variety of mechanisms for achieving linear translation of the adjustable foam sensor 82. For example, actuator 350 can include mechanical, electro-mechanical, hydraulic, pneumatic, piezoelectric, etc.

In some embodiments, the actuator 350 can be a rotary actuator such that the actuator 350 causes the adjustable foam sensor 84 to rotate about an axis. For example, the adjustable foam sensor 84 can be shaped such that rotation of the adjustable foam sensor 84 results in a horizontal and/or vertical displacement of the foam probe 92 within the chamber 32.

FIG. 6 depicts an embodiment of the adjustable foam sensor system 580 including two exemplary adjustable foam sensor assemblies 582A-B integrated with a container 532 that can have the same or similar features and functions as container 32 of FIG. 1. For example, the container 532 can be a bioprocess container for carrying out biological processes that use or yield biological reagents or end-products including, but not limited to, carbohydrate, cell, cell media, drug, enzyme, lipid, nucleic acid (e.g., DNA, RNA), pharmaceutical, plasmid, protein, reagent, vaccine, viral vector and virus end-products. The container 532 can include container ports 533A-533C for interfacing with and attaching to adjustable foam sensor assemblies 582A-B and/or other devices, such as an anti-foam delivery device 54 (as shown in FIG. 1) coupled to container port 533B. The container ports 533A-533C can be any one of the container ports 33A-33F described with respect to FIG. 1. One or more adjustable foam sensor assemblies 582A-B can be hermetically integrated with the container 532 to provide additional versatility and to detect foam in a variety of vertical, radial and lateral positions within the container 532.

The adjustable foam sensor assemblies 582A-B can each include an adjustable foam sensor 584A-B with an adjustable sensor housing 587 that facilitates the free movement of the adjustable foam sensors 584A-B into a variety of positions within the container 532. Each adjustable foam sensor 584A-B includes a sensor base 590, a container interface 589, one or more foam probes 592A-592D, a transition member 594 and a sensor probe guide 501. The container interface 589 can attach to, connect and/or otherwise interface with the container port 533 to create a hermetic seal between the container interface 589 and container 532. The container interface 589 can be a flange of any shape, including an annular flange 589 with one or more openings 586 through which the transition member 594 of the adjustable foam sensors 584A-B are received and disposed. The transition member 594 can freely move through, in and out of the opening 586 during use, extension, and retraction of the adjustable foam sensors 584A-B. The container interface 589 connects or otherwise attaches to the container 532 at a port 533 formed in the container 532 to create an air-tight and/or liquid-tight seal. In an exemplary embodiment, the container interface 589 can include a ½ inch face port opening 586 or other size opening (e.g. up to 2 inches).

In exemplary embodiments, the adjustable sensor housing 587 can include a housing body 585 and a housing base 599. The housing body 585 of the adjustable sensor housing 587 can be formed from pliable and flexible bellows that can be compressed and extended like a spring. The housing base 599 can be formed from one or more rigid materials capable of absorbing forces (e.g., pull and push forces) and transferring those forces to the housing body 585 of the adjustable sensor housing 587. One end of the housing body 585 attaches to, connects to and/or otherwise interfaces with the container interface 589, and the other end of the housing body 585 connects to and/or otherwise interfaces with housing base 599. In exemplary embodiments, the housing base 599 can be attached to the sensor base 590 by barbed, threaded, quick connect adaptor or weld that can form an aseptic seal between the two parts.

To actuate, move, extend or retract the adjustable foam sensors 584A-B partly and/or completely in and out of the container 532, a force can be applied to the housing base 599, causing the adjustable sensor housing 587 to expand or compress. For example, the adjustable sensor housing 587 can be shortened, retracted and/or compressed by applying a push force to the housing base 599 while retaining the container interface 589 in a fixed or substantially fixed position. Likewise, the adjustable sensor housing 587 can be stretched, lengthened and/or extended by applying a pull force to the housing base 599 while retaining the container interface 589 in a fixed or substantially fixed position. The container interface 589 of the adjustable foam sensor 584 can be a fixed or substantially fixed to the container 532 to facilitate actuation of both the adjustable sensor housing 587 and the adjustable foam sensor 584.

Other forces can also be applied to the adjustable sensor housing 587 and other housing configurations can also be used to actuate both the adjustable foam sensor housing 587 and adjustable foam sensors 584A-B and position the foam probe 592 at distinct locations within the container 532.

For example, as shown in FIG. 6, a first actuator 350A and a second actuator 350B can be used to each apply either push or pull forces along an associated housing base 599 of an adjustable foam sensor assembly 582A-B. This can cause the associated adjustable sensor housing 587 to expand or compress, as well as move the foam probe in the container 532. For example, the first actuator 350A can be coupled to the housing base 599 of the adjustable foam sensor assembly 582A coupled to a top portion of the container 532 and the second actuator 350B can be coupled to the housing base 599 of the adjustable foam sensor assembly 582A coupled to a sidewall of the container 532.

During use and to move the adjustable foam sensors 584A-B partly and/or completely in and out of the container 532, a force from the associated actuator 350A-B can be applied to the adjustable sensor housing 587 to extend and compress the housing 587. For example, after securing the container interface 589 of the adjustable foam sensor 584 to the container 532, a push force applied to the housing base 599 also causes and applies a push force to the adjustable foam sensor base 590, which in-turn causes the flexible bellows of the housing body 585 to compress and the one or more foam probes 592A-D and/or a portion of the transition member 594 to extend and/or advance into the container 532. Likewise, a pull force applied to the housing base 599 also causes and applies a pull force to the adjustable foam sensor base 590, which in-turn causes the flexible bellows of the housing body 585 to extend and the one or more foam probes 592A-D and/or a portion of the transition member 594 to retract and/or move in a direction to retract from the container 532.

As shown in FIG. 6, the adjustable foam sensor system 580 can include an embodiment of the controller 336 in communication with a fluid sensing component, such as a mass measuring sensor 550 (e.g., load cell) and/or a pressure measuring sensor 552. The mass measuring sensor 550, for example, can be coupled to the container 532 and measure a mass of the container 532 and fluid 541. The mass of the fluid 541 can be determined if the mass of the container 532 is known, and a fluid level 575 can be determined if the dimensions of the container 532 are known. Additionally or alternatively, the fluid level 575 can be determined using the pressure measuring sensor 552. For example, the fluid level 575 can be determined based on known dimensions of the container 532 and sensed pressure readings of the fluid 541, such as pressure readings taken from the pressure sensor 552 positioned along a bottom surface of the container 532, as shown in FIG. 6. The fluid sensing components, such as the mass measuring sensor 550 and/or the pressure measuring sensor 552, can continuously and/or intermittently communicate with the controller 336 and continuously and/or intermittently provide the controller 336 with the sensed fluid data (e.g., the sensed mass and/or pressure data).

The controller 336 can process the sensed fluid data (e.g., using at least the processor 337) for determining a current fluid level 575 in the container 532. For example, the controller 336 can determine if a change in fluid level in the container 532 has occurred, such as by comparing the current fluid level with a fluid parameter, such as a previously determined or saved fluid level. As such, the controller can determine if the fluid level 575 has increased (e.g., risen) or decreased (e.g., fallen) and then, based on a determined change in fluid level 575, the controller can automatically or approximately automatically activate at least one of the actuators 350A-B to move an associated adjustable foam sensor 584. As such, one or both the actuators 350A-B can be activated, based on the determined change in fluid level 575, to thereby move the adjustable foam sensors 584A-B so that the foam probes 592A-D are positioned in desired locations relative to the current fluid level 575 (e.g., at least one foam probe 592A-D is positioned above and adjacent the top surface 376 of the fluid 541) in order to efficiently and effectively detect foam in the container 532. For example, the controller 336 can control one or both actuators 350A-B such that they move the foam probe in a vertical direction and a vertical distance that is based on the determined change in fluid level 575. As such, if the fluid level 575 is determined to have increased by approximately 4 inches, the controller 336 can control the actuators 350A-B to directly or indirectly move the foam probes 592A-D approximately 4 inches (e.g., in a vertical direction) within the container 532. Such monitoring of fluid levels 575 and automatically controlling the positioning of the foam probes 592A-D based on determined changes in fluid levels 575 can allow the adjustable foam sensor system 580 to efficiently and effectively monitor foam formation, as well as efficiently and effectively reduce and/or eliminate foam in the container 532. Furthermore, once a foam probe 592A-D senses foam in the container 532, the controller 336 can activate an embodiment of the anti-foam dispenser in fluid communication with the container 532 (such as anti-foam dispenser 54 in FIG. 1) in order to dispense a volume of anti-foam in the container 532 for suppressing and/or eliminating foam in the container 532.

In FIG. 6, two adjustable foam sensor assemblies 582A-B can be hermetically coupled to the container 532 through container ports 533A and 533C. One adjustable foam sensor assembly 584B can be mounted to the sidewall of the container 532, and the other adjustable foam sensor assembly 582A can be mounted to the top wall of the container 532, as shown in FIG. 6. The side-mounted adjustable foam sensor assembly 582B can have an L-shaped transition member 594 and a foam probe 592D positioned at a distal end of the L-shaped transition member 594. In some embodiments, the transition member 594 is formed of a rigid material and/or a flexible material. As such, the L-shaped transition member 594 can be formed of a flexible material such that the foam probe 592D is vertically raised and lowered based on the approximately horizontal translation of, for example, a proximal end of the transition member 594. For example, as the proximal end of the flexible L-shaped transition member 594 is horizontally translated or moved in a first direction (e.g., away from the inside of the container 532), the foam probe 592D can be raised (e.g., in a vertical direction). In some embodiments, the L-shaped transition member 594 can be rigid and coupled to a rotary actuator that rotates the L-shaped transition member 594 to thereby move the position of the foam probe 592D within the container 531, such as in response to a determined change in fluid level 575. The top-mounted adjustable foam sensor assembly 582A can include three foam probes 592A-C extending from a node of the transition member 594, including an L-shaped transition member 594, a vertical foam probe 592B facing down and a lateral foam probe 592A facing towards a sidewall of the container 532. A rigid casing 500 or probe housing can house and support transition member 594 and foam probe 592D in the L-shaped position, and guide and center the transition member 594 and foam probe 592D to prevent unwanted contact with surfaces and prevent signal fouling.

The side-mounted adjustable foam sensor assembly 582B and the top-mounted adjustable foam sensor assembly 582A alone or together can detect the presence of foam at multiple vertical and lateral positions in the container 532, which can allow the adjustable foam sensor system 80 to model and predict the dynamics of foam build-up and enhance the granularity of foam detection and suppression. Signals and data indicative of foam concentration, density and/or volume sent from one or more foam probes 595A-D at multiple positions within the container can be processed at the controller 336, that also controls a foam suppression system and dispenser (e.g., anti-foam dispenser 54 of FIG. 1) for releasing foam suppressant or anti-foaming agent into the container 532.

For example, the side-mounted adjustable foam sensor 584B with L-shaped transition member 594 and foam probe 592D can be extended and retracted horizontally across the container 532 to detect foam at different horizontal positions across the top surface 576 of the process fluid 541 in the container 532. The side-mounted adjustable foam sensor 584B can also be rotated to detect foam at higher levels in the container 532. The L-shaped transition member 594 and foam probe 592D of the side-mounted adjustable foam sensor 584D can include a void space surrounding the transition member 594 and/or foam probe 592 to prevent moisture collecting and running down sidewall from contacting the transition member 594 to reduce fouling.

The top-mounted adjustable foam sensor 584A with L-shaped, lateral and vertical directed foam probes 592A-C can be extended and retracted vertically up and down the container 532 to detect foam at different vertical positions at and/or above the top surface 576 of the process fluid 541 in the container 532. The top-mounted adjustable foam sensor 584A can also be rotated to detect foam at radial positions in an inflated and three-dimensional container 532 with the lateral and L-shaped transition member 594 with foam probes 592A, 592C respectively.

In exemplary embodiments, any number of adjustable foam sensor assemblies 582 can be mounted to a bioprocess container 532 to detect foam in a variety of locations within the container 532. In addition, any number and shape of foam probes 592 can extend from a node in the transition member 594 of an adjustable foam sensor 284 to detect foam in a variety of locations within the container 532. Multiple adjustable foam sensor assemblies 582 and/or foam probes 592 can provide more data to model, track and suppress foam in a container 532 or other fluid processing equipment.

In exemplary embodiments, the transition member 594, foam probes 592A-D, or both can be formed into any shape, including L-shapes. Together or independently, transition member 594 and foam probes 592A-D can be rigid and/or flexible members forming any shape.

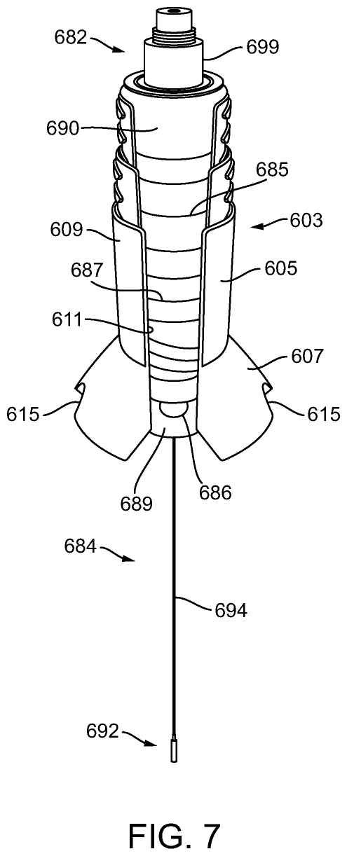

FIG. 7 depicts an exemplary adjustable foam sensor assembly 682 including a foam sensor holder 603. The adjustable foam sensor assembly 682 can include the same or similar components and functionality as the adjustable foam sensor assemblies discussed above with respect to, for example, FIGS. 1-6. The adjustable foam sensor assembly 682 includes an adjustable foam sensor 684 with an adjustable sensor housing 687 that facilitates movement of the adjustable foam sensor 684 into a variety of positions within a container (e.g., container 32 of FIG. 1), such as a bioprocess container. The adjustable foam sensor 684 includes a sensor base 690, a container interface 689, one or more foam probes 692, a transition member 694 and a sensor probe guide (shown as 401 and 501 in FIGS. 4A and 6). The container interface 689 can attach to, connect and/or otherwise interface with a container port to create a hermetic seal between the container interface 689 and container. The container interface 689 can be a flange of any shape, including an annular flange 689 with one or more openings 686 through which the transition member 694 and foam probe 692 of the adjustable foam sensor 684 are received and disposed. The transition member 694 can freely move through, in and out of the opening 686 during use, extension and retraction of the adjustable foam sensor 684. The container interface 689 connects or otherwise attaches to a container to create an air-tight and/or liquid-tight seal.

In exemplary embodiments, the adjustable sensor housing 687 can include a housing body 685 and a housing base 699. The housing body 685 of the adjustable sensor housing 687 can be formed from pliable and flexible bellows that can be compressed and extended like a spring. The housing base 699 can be formed from one or more rigid materials capable of absorbing forces (e.g., pull and push forces) and transferring those forces to the housing body 685 of the adjustable sensor housing 687. One end of the housing body 685 attaches to, connects to and/or otherwise interfaces with the container interface 689, and the other end of the sensor housing body 685 connects to and/or otherwise interfaces with housing base 699. In exemplary embodiments, the housing base 699 can be attached to the sensor base 690 by barbed, threaded, quick connect adaptor or weld that can form an aseptic seal between the two parts, however, mechanisms are with the scope of this disclosure.

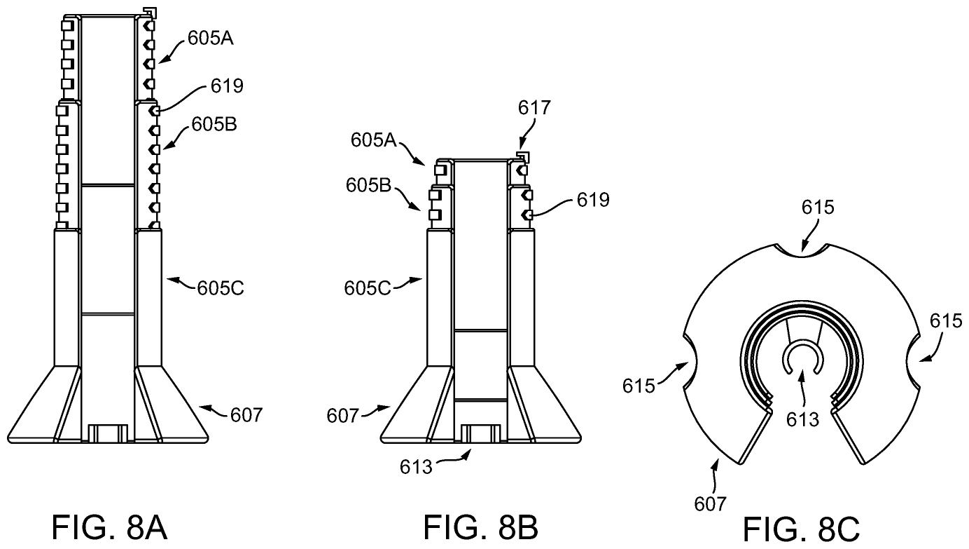

FIGS. 8A-8C depict an exemplary foam sensor holder 603. Referring collectively to FIGS. 7, 8A-8C, the sensor holder 603 includes a sensor holder body 605 and sensor holder base 607. The sensor holder body 605 can include an external surface 609 and internal surface 611. The internal surface 611 of the sensor holder body 605 can include retainment elements on the internal surface, including but not limited to, grooves, slots, protrusions, notches or pins that protrude or provide an opening to engage with one or more engagement elements (e.g., one or more grooves, slots, protrusions, notches or pins) along an outer surface of the sensor housing body 685.

The sensor holder body 605 can be a telescoping body, tube or other shaped body with a tapered geometry and a variable radius along its longitudinal axis. In this embodiment, each section of the telescoping body 605A-B (shown in FIGS. 8A-8B) has a different radius and can have a sliding engagement with at least one adjacent telescoping body, such as telescoping body 605B can have a sliding engagement, and section A can collapse and slide into section B, and section B combined with section A can collapse and slide into section C, such as similar to a telescope. FIG. 8A illustrates the sensor holder 603 and sensor holder body 605 in the extended state, and FIG. 8B illustrates the sensor holder 603 and sensor holder body 605 in the collapsed state. In some embodiments, one or more segments of the sensor holder body 605A-C can include one or more engagement elements 619 that can engage with complimenting engagement features along an adjacent sensor holder body 605 segment. For example, the engagement elements 619 can include extruded threads, slots, pins, etc., that allow sliding and/or rotating engagement between the segments of the sensor holder body 605 and securing relative positions.

The sensor holder base 607 (also shown in FIG. 8C) can include one or more indentions 615 that can interface with retention elements, such as clips, protrusions or inserts, on the container to couple and lock the sensor holder 603 to the container. In addition to indentions 615, the sensor holder base 607 can include a securing element 613 that engages and couples to a surface of the container or container port to fix the foam sensor holder 603 to the container. The top of the sensor holder body 605 can also include a securing element 617 that engages and couples to another surface, frame, mount or other component to secure the foam sensor holder 603 in place vertically or horizontally to a bioprocess container (shown in FIG. 6).

The sensor housing body 685, including the adjustable foam sensor assembly 682, can be inserted, routed through, and/or press-fit into the sensor holder body 605. The sensor holder body 605 can include retainment elements (e.g., one or more extrusions, extruded threads, slot, etc.) on the internal surface that engage and interface with one or more surfaces of the sensor housing body 685 to couple the housing 687 to the holder 603 and lock the adjustable foam sensor assembly 682 into various extended and retracted positions. In other embodiments, retainment elements are not necessary and the contact and fit between the internal surface 611 of the sensor holder 603 and the sensor housing body 685 fixes and/or couples the sensor housing body 685 to the sensor holder body 605.

After inserting the adjustable foam sensor assembly 682 into the sensor holder body 605, the adjustable foam sensor assembly 682, including sensor housing body 685, transition member 694 and foam probe 692, can be retracted and extended by collapsing or extending the telescoping body 605A-B of the sensor holder 603. The press-fit and contact between the internal surface 611 of the sensor holder 603 and the sensor housing body 685 also forces the sensor housing body 685 to retract and extend. In other embodiments, a force can be applied to the sensor housing base 699, as previously discussed, until the sensor housing body 685 engages a retainment element (not shown) that locks the sensor holder body 605 and the adjustable foam sensor assembly 682 into a specific retracted or extended state.

In exemplary embodiments, telescoping body 605A-B of the adjustable sensor holder 603 can be collapsed to compress the sensor housing body 687 and extend the transition member 694 and foam probe 692 into a container (shown in FIG. 6). The telescoping body 605A-B of the sensor holder 603 can also be extended to extend the sensor housing body 687 and retract the transition member 694 and foam probe 692 inside the container (shown in FIG. 6).

Like the assemblies illustrated in FIG. 6, the adjustable foam sensor assembly 682 including the adjustable foam sensor holder 603 of FIGS. 7-8C can be mounted to a top wall, sidewall or bottom wall of a container to detect foam at multiple positions in a bioprocess container.

FIG. 9 illustrates communication between hardware of an adjustable foam sensor system 80 according to exemplary embodiments of the present disclosure. The hardware and communication between hardware shown in FIG. 9 can be included in any of the adjustable foam sensor systems 80, 380, 580 disclosed herein and/or within the scope of this disclosure. For example, embodiments of the adjustable foam sensor system 80 can include a controller 336 including and/or in communication with a processor 337. The adjustable foam sensor system 80 can also include foam sensing devices, such as an embodiment of the adjustable foam sensor assembly 84 including a foam probe 92 (as shown in FIG. 1), as well as a ground contact 146 and probe 148 (as also shown in FIG. 1). The foam sensing devices can be configured to detect the presence of foam in the fluid processing system 10 (as shown in FIG. 1) and, as a result of detecting foam, can generate a sensed foam data that is indicative of foam being present within the container 32 (as shown in FIG. 1). Such sensed foam data can be sent to and retrieved by the controller 336.

The adjustable foam sensor system 80 can also include a fluid measuring device, such as a mass measuring sensor 550 and/or a pressure measuring sensor 552. The fluid measuring device can collect and/or generate measured data, such as measured mass and/or pressure data. For example, the controller 336 can receive the measurement mass data and/or measurement pressure data and use such data to determine a fluid level of the fluid in the container (FIGS. 1 and 6). The controller 336 can compare the determined fluid level of the fluid against a fluid parameter, such as a previously determined fluid level and/or a saved fluid level (e.g., threshold level, etc.). For example, if the controller 336 determines a difference between the current determined fluid level and the fluid parameter, the controller can send an instruction to the foam sensor positioning mechanism (e.g., actuator 350 including linear and/or rotary actuator) to move the foam probe 92, such as approximately a same or similar distance as the difference between the fluid parameter and the current fluid level. After the foam probe 92 has been correctly positioned within the container for effectively determining the presence of foam in the container 32, the foam probe 92 can contact foam and generate sensed foam data indicating the presence of foam in the container 32. The controller 336 can receive the sensed foam data and, when the sensed foam data indicates the presence of foam in the container 32, the controller 336 can send instructions and/or activate the anti-foam dispenser (e.g., for dispensing anti-foam solution in the container 42) and/or control one or more mechanical elements (e.g., reduce and/or turn-off sparger 66 and/or mixer drive shaft 68 and impeller 70 (as shown in FIG. 1)) that can reduce and/or eliminate foam. As such, the adjustable foam sensor system 80 can be fully or at least partially automated for efficiently and effectively repositioning foam probes relative to changing fluid levels in a container, detecting foam in the container, and reducing and/or eliminating foam in the container. Other hardware and communication lines between hardware and devices of the adjustable foam sensor system 80 are within the scope of this disclosure.

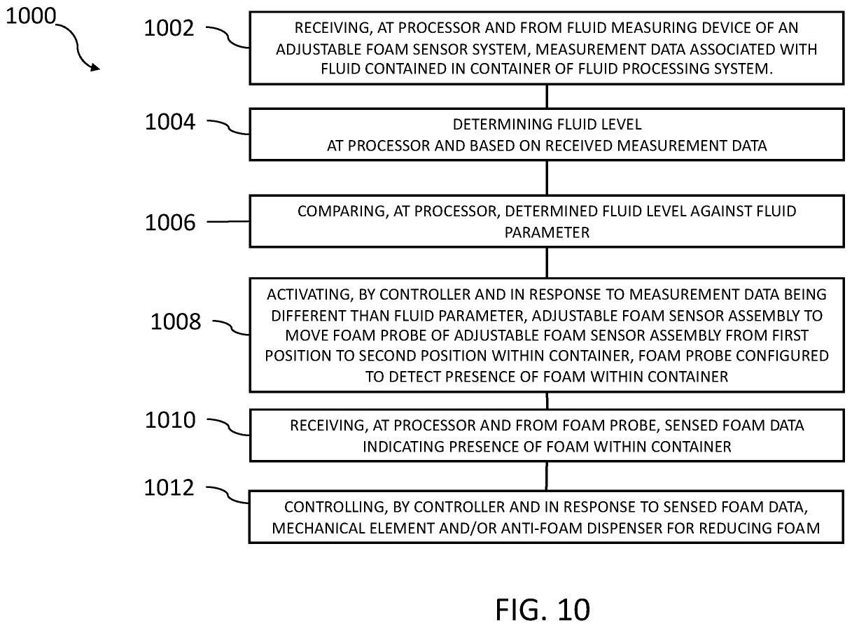

FIG. 10 illustrates a foam detection and reduction process 1000 according to exemplary embodiments of the present disclosure. The foam detection and reduction process 1000 can be performed by an adjustable foam sensor system 80 (as shown in FIG. 1) including any of the embodiments of the adjustable foam sensor system 80 and components thereof described herein, such as with regards to at least FIGS. 1-9. The foam detection and reduction process 1000 can be performed at a controller 336 including a processor 337 (FIG. 9) and non-transitory storage medium (e.g., of controller 336) of the adjustable foam sensor system 80 and/or performed at a controller and/or processor separate from the adjustable foam sensor system 80.

The adjustable foam sensor assembly can include an adjustable foam sensor 84 including a transition member 94 (FIGS. 2A and 2B) with a foam probe 92 extending from the transition member 94. The foam probe 92 can detect a presence of foam within a container 32 of the fluid processing system 10 (as shown in FIG. 1), and the transition member 94 can extend through an interface opening along the chamber 32 and move along the interface opening in response to a force applied to the adjustable foam sensor 84 to thereby move the foam probe 92 from a first position to a second position to more efficiently and effectively sense and reduce foam in the container 32.

At 1002, the processor 337 can receive measurement data from a fluid measuring device (e.g., mass measure sensor 550 and pressure measuring sensor 552, as shown in FIG. 6) of an adjustable foam sensor system 80. The measurement data can be associated with a fluid 41 contained in the container 32 of the fluid processing system 10.

At 1004, a fluid level 575 (as shown in FIG. 6) can be determined at the processor 337 and based on the received measurement data.

At 1006, the determined fluid level 575 can be compared against a fluid parameter (e.g., a previously determined fluid level) at the processor 337.

At 1008, the controller 336 can activate, in response to the determined fluid level being different than the fluid parameter, an adjustable foam sensor assembly 82 (as shown in FIGS. 2A and 2B) to move the foam probe 92 (configured to detect a presence of foam within the container) from a first position to a second position within the container 32.

At 1010, sensed foam data can be received by the processor 337 indicating the presence of foam within the container 32.

At 1012, the controller 336 can control, in response to the sensed foam data, an anti-foam dispenser to deliver a volume of anti-foam solution into the container for reducing the foam.

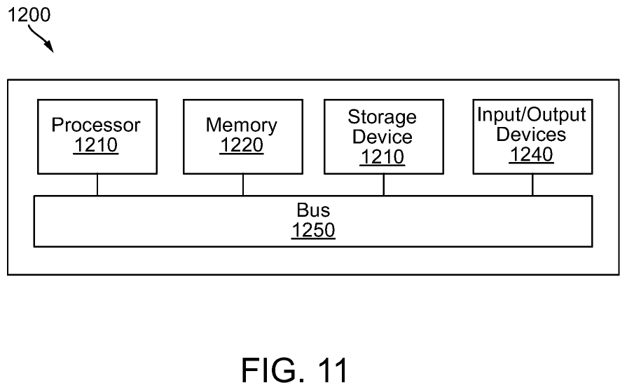

FIG. 11 illustrates a block diagram depicting an example of a computing system consistent 1200 with implementations of the current subject matter.

The computing system 1200 can be used to implement the adjustable foam sensor system and/or any component therein. For example, the computing system 1200 can implement user equipment, a personal computer, or a mobile device.

As shown in FIG. 11, the computing system 1200 can include a processor 1210, a memory 1220, a storage device 1230, and an input/output device 1240. The processor 1210, the memory 1220, the storage device 1230, and the input/output device 1240 can be interconnected via a system bus 1250. The processor 1210 is capable of processing instructions for execution within the computing system 1400. Such executed instructions can implement one or more components of, for example, the foam layer measuring system 301 for calculating a thickness 311 of the foam layer 350 and determining whether a foam layer threshold is satisfied. In some example embodiments, the processor 1210 can be a single-threaded processor. Alternately, the processor 1210 can be a multi-threaded processor. The processor 1210 is capable of processing instructions stored in the memory 1220 and/or on the storage device 1230 to display graphical information for a user interface provided via the input/output device 1440.

The memory 1220 is a non-transitory computer-readable medium that stores information within the computing system 1200. The memory 1220 can store data structures representing configuration object databases, for example. The storage device 1230 is capable of providing persistent storage for the computing system 1200. The storage device 1230 can be a floppy disk device, a hard disk device, an optical disk device, or a tape device, or other suitable persistent storage means. The input/output device 1240 provides input/output operations for the computing system 1200. In some example embodiments, the input/output device 1240 includes a physical or virtual keyboard and/or pointing device. In various implementations, the input/output device 1240 includes a display unit for displaying graphical user interfaces. The display unit can be a touch activated screen that displays and facilitates user input/output operations.

According to some example embodiments, the input/output device 1240 can provide input/output operations for a network device. For example, the input/output device 1240 can include Ethernet ports or other networking ports to communicate with one or more wired and/or wireless networks (e.g., a local area network (LAN), a wide area network (WAN), the Internet, a public land mobile network (PLMN), and/or the like). Other communication protocols can include analog, digital and/or other communication signals.

In some example embodiments, the computing system 1200 can be used to execute various interactive computer software applications that can be used for organization, analysis, and/or storage of data in various formats. Alternatively, the computing system 1200 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing items and/or functionalities. Upon activation within the applications, the functionalities can be used to generate the user interface provided via the input/output device 1240. The user interface can be generated and presented to a user by the computing system 1200 (e.g., on a computer screen monitor, etc.).

Various alterations and/or modifications of the inventive features illustrated herein and additional applications of the principles illustrated herein, which would occur to one skilled in the relevant art and having possession of this disclosure, can be made to the illustrated embodiments without departing from the spirit and scope of the invention as defined by the claims and are to be considered within the scope of this disclosure. Thus, while various aspects and embodiments have been disclosed herein, other aspects and embodiments are contemplated. While several methods and components similar or equivalent to those described herein can be used to practice embodiments of the present disclosure, only certain components and methods are described herein.

It will also be appreciated that systems, processes, and/or products according to certain embodiments of the present disclosure may include, incorporate, or otherwise comprise properties features (e.g., components, members, elements, parts, and/or portions) described in other embodiments disclosed and/or described herein. Accordingly, the various features of certain embodiments can be compatible with, combined with, included in, and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include said features without necessarily departing from the scope of the present disclosure.

The present disclosure may be embodied in other specific forms without departing from its spirit or essential characteristics. The described embodiments are to be considered in all respects only as illustrative and not restrictive. The scope of the invention is, therefore, indicated by the appended claims rather than by the foregoing description. While certain embodiments and details have been included herein and in the attached disclosure for purposes of illustrating embodiments of the present disclosure, it will be apparent to those skilled in the art that various changes in the methods, products, devices, and apparatus disclosed herein may be made without departing from the scope of the disclosure or of the invention, which is defined in the appended claims. All changes which come within the meaning and range of equivalency of the claims are to be embraced within their scope.

1. An adjustable foam sensor assembly comprising:
a container interface comprising an interface opening, the container interface configured to couple to a container of a fluid processing system; and
an adjustable foam sensor comprising a transition member with a first foam probe extending from the transition member, wherein the first foam probe detects a presence of foam within the container, and wherein the transition member extends through the interface opening and moves along the interface opening in response to a force applied to the adjustable foam sensor to thereby move the first foam probe from a first position to a second position.

2. The adjustable foam sensor assembly of claim 1, further comprising:
an adjustable housing configured to transition between a compressed configuration and an extended configuration, the compressed configuration positioning the first foam probe in the first position and the extended configuration positioning the first foam probe in the second position that is a distance from the first position.

3. The adjustable foam sensor assembly of claim 2, further comprising:
a sensor probe guide within the adjustable housing that retains and centers the transition member in the interface opening, the sensor probe guide comprising a guide opening through which the transition member extends.

4. The adjustable foam sensor assembly of claim 3, wherein the transition member linearly translates through the guide opening in response to the applied force.

5. The adjustable foam sensor assembly of claim 1, wherein the transition member is coupled to a linear actuator.

6. The adjustable foam sensor assembly of claim 1, wherein the transition member is coupled to a rotary actuator.

7. The adjustable foam sensor assembly of claim 1, wherein the transition member is L-shaped.

8. The adjustable foam sensor assembly of claim 1, further comprising a second foam probe extending from the transition member.

9. The adjustable foam sensor assembly of claim 8, further comprising a third foam probe extending from the transition member.

10. The adjustable foam sensor assembly of claim 1, wherein the transition member is formed of a flexible material.

11. The adjustable foam sensor assembly of claim 1, wherein the transition member is formed of a rigid material.

12. The adjustable foam sensor assembly of claim 8, wherein the first foam probe is configured to communicate with a controller and provide sensed foam data to the controller when the first foam probe is in contact with foam, the sensed foam data indicating the presence of foam within the container.

13-15. (canceled)

16. The adjustable foam sensor system of claim 1, further comprising:
an anti-foam dispenser coupled to a second port of the container for dispensing anti-foam into the container.

17. The adjustable foam sensor system of claim 16, further comprising:
a fluid measuring device that is at least one of coupled to the container and in communication with the fluid for generating measurement data for determining a fluid level in the container.

18. The adjustable foam sensor system of claim 17, wherein the fluid measuring device is a mass measuring sensor or a pressure measuring sensor

19-29. (canceled)

30. A method of reducing foam in a container comprising:
at least partially filling a container with a fluid, the container comprising a first container port coupled to an adjustable foam sensor assembly, the adjustable foam sensor assembly comprising:
a container interface comprising an interface opening, the container interface configured to couple to the container; and
an adjustable foam sensor comprising a transition member with a foam probe extending from the transition member, wherein the foam probe senses a presence of foam within the container, and wherein the transition member extends through the interface opening and moves along the interface opening in response to a force applied to the adjustable foam sensor to thereby move the foam probe from a first position to a second position; and
applying a force to the transition member to move the foam probe from the first position to the second position that is above and adjacent a top surface of the fluid.

31. The method of claim 30, further comprising:
detecting, by the foam probe, the presence of foam within the container.

32. The method of claim 31, further comprising:
receiving, at a controller in communication with the foam probe, a signal indicative of the presence of foam.

33. The method of claim 32, further comprising:
activating, by the controller, an anti-foam dispenser coupled to a second container port to dispense a volume of antifoaming agent into the container to reduce the foam.

34. The method of claim 31, wherein the detecting the presence of foam within the container comprises continuously sending, from the controller, a signal to the foam probe and identifying a change in the signal indicative of the presence of foam.

35-42. (canceled)
